(19) United States
(12) Patent Application Publication
(10) Pub. No.: US 2026/0263394 A1
(43) Pub. Date: Sep. 10, 2026

(54) MEDICAL PATCH COMPRISING SKIN IRRITATING ACTIVE AGENT

(71) Applicant: LTS LOHMANN THERAPIE-SYSTEME AG, Andernach (DE)

(72) Inventor: Bernd GRÜNES, Bonn (DE)

(21) Appl. No.: 19/164,677

(22) PCT Filed: Mar. 15, 2024
(86) PCT No.: PCT/EP2024/057009

§ 371 Date: Sep. 12, 2025

(30) Foreign Application Priority Data
Mar. 15, 2023 (EP) 23162161.6
Mar. 15, 2023 (EP) 23162162.4
Apr. 28, 2023 (EP) 23170588.0

(51) Int. Cl. A61K 31/165; A61K 9/70; A61K 31/352; A61K 47/34

(52) U.S. Cl. CPC A61K 31/165; A61K 9/7084; A61K 31/352; A61K 47/34

(57) ABSTRACT

The present invention relates to a medical patch for the administration of an active agent comprising an active agent-containing layer structure, said active agent-containing layer structure comprising a backing layer, an active agent-containing layer and a skin contact layer, wherein the skin contact layer is an adhesive layer which is directly attached to the active agent-containing layer, and wherein the saturation concentration of the active agent in the skin contact layer is less than 0.1% by weight, as well as to such a medical patch for use in a method of treatment and to a process of manufacture of such a medical patch.

### TECHNICAL FIELD OF THE INVENTION

The present invention relates to a medical patch for the administration of an active agent, wherein the medical patch comprises an active agent-containing layer and a skin contact layer, and wherein the saturation concentration of the active agent in the ski contact layer is less than 0.1% by weight. Further, the present invention relates to processes of manufacture as well as methods of treatment and uses of the medical patch.

### BACKGROUND OF THE INVENTION

The most common routes of drug delivery are the oral and parenteral routes with the majority of small molecule drugs conventionally delivered orally. The oral route has the advantage of pre-determined doses, portability and the possibility of patient self-administration but often correlates with gastrointestinal adverse effects. Parenteral administration such as intravenous or subcutaneous administration is not without limitations, such as the invasive nature of injections eliciting pain and lower acceptance/compliance by patients, in addition to the requirement for administration by a trained administrator.

On the other hand, the skin offers an accessible and convenient site for non-invasive administration of medications, e.g., by using medical patches placed on the skin of a patient to deliver a specific dose of medication through the skin.

Medications have been topically applied to the skin for a long time to treat local conditions, while more recently, transdermal delivery technology has been developed to treat a range of conditions beyond the local site of application. While topical delivery of a compound and/or drug is limited to treat conditions locally and avoids systemic effects, transdermal medications refer to pharmaceutical compounds that are applied on the skin and cross not only the stratum corneum but also the epidermis to reach the blood stream and/or for targeting an effect on more distant tissues or organs.

Drug delivery through the skin has many advantages over other conventional routes of medication. First, the skin, being the most extended and most easily accessible organ of the body, allows many placement options for medical patches. Drug delivery through the skin can thus provide a non-invasive alternative to parenteral routes, circumventing issues such as needle phobia. In addition, it can improve patient compliance due to the reduction of dosing frequencies and is also suitable for patients who are unconscious or vomiting, or those who rely on self-administration. Second, drug delivery through the skin avoids pre-systemic metabolism, thus improving bioavailability. Furthermore, the pharmacokinetic profiles of drugs are usually more uniform with fewer peaks, thus minimizing the risk of side effects due to peak concentrations.

On the other hand, the drug, the adhesive or other excipients contained in the medical patch may be an irritating substance which provokes application site reactions including skin irritation such as irritant contact dermatitis (ICD) or allergic contact dermatitis (ACD). ACD is a type IV cell-mediated hypersensitivity reaction that usually presents with lesions that can vary from erythema and papules to vesicles and bullae. ICD develops when the drug, adhesive or excipient damage the skin surface faster than the skin can repair, presenting skin lesions similar to ACD ones. Common application site signs and symptoms include localized redness (erythema) or itching, sometimes accompanied by swelling (edema). Typically, these are mild to moderate in severity and transient in nature. Most are localized to the area of application, and resolve spontaneously within several days following patch removal. However, some medical patches may even cause systemic reactions and/or pain depending on the irritating substance contained therein.

In fact, the occurrence of such (skin) reactions are not limited to the application site, as contamination with the irritating substance can already occur during application of the medical patch. In particular, the irritating substance can get into contact with the fingers or other parts of the hands of the person applying the medical patch, with the risk of subsequently being transferred to other particularly sensitive areas of the body such as the eyes. Due to this risk coming along with skin irritating medical patches and in particular skin irritating active agents contained in medical patches, application of the same requires appropriate precautions and is often limited to medical professionals only, depriving the medical patch of the advantage of (otherwise possible) self-administration by the patient.

It is therefore desirable to provide a medical patch for the administration of an (irritating) active agent, which allows for safe and uncomplicated handling. In particular, there is a need for a medical patch, avoiding contamination with the active agent contained therein, even in case of unawareness, negligence, carelessness, inattentiveness and/or lack of skill of the person applying the medical patch.

### OBJECTS AND SUMMARY OF THE INVENTION

It is an object of the present invention to provide a medical patch overcoming the disadvantages of current administration of irritating active agents.

It is a further object of the present invention to provide a medical patch for the administration of an active agent, which can be applied/removed in a safe and uncomplicated manner. In particular, the object is to provide a medical patch for the administration of an active agent, which is allowed to be safely applied/removed by a patient or other person not being medical professional.

It is a further object of the present invention to provide a medical patch for the administration of an active agent, which avoids substantial contamination with the active agent. In particular, the object is to provide a medical patch for the administration of an irritating active agent, requiring only few or no precautions during the application/removal of the medical patch.

It is a further object of the present invention to provide a medical patch for the administration of an active agent, which reduces the problem of unwanted skin reactions.

It is also an object of the present invention to provide a medical patch for the administration of an active agent, providing a permeation rate which is sufficient for achieving a therapeutically effective dose and having improved adhesive properties.

It is another object of the present invention to provide a medical patch for the administration of an active agent, which can be used in a method of treatment.

These objects and others are accomplished by the present invention, which according to one aspect relates to a medical patch for the administration of an active agent comprising an active agent-containing layer structure, said active agent-containing layer structure comprising:
A) a backing layer;
B) an active agent-containing layer comprising
   (i) an active agent, and
   (ii) a polymer I;
   and
C) a skin contact layer comprising a polymer II;
   wherein the skin contact layer is an adhesive layer which is directly attached to the active agent-containing layer, and
   wherein the saturation concentration of the active agent in the skin contact layer is less than 0.1% by weight.

It has been surprisingly found that a medical patch according to the present invention, which comprises a skin contact layer, in which the active agent is sparingly soluble, is still able to provide a sufficient drug delivery. Since the active agent is sparingly soluble, e.g., has a saturation concentration of less than 0.1% in the skin contact layer, only an insignificant amount of active agent is present on the surface of the skin contact layer so that the medical patch has advantageous properties regarding unwanted skin reactions and thus enables safe application and/or removal. Thus, such an inventive medical patch with a skin contact layer, in which the saturation concentration of the active agent is negligible, with a separated active agent-containing layer (not being in skin contact), prohibits the active agent to be released before and/or after the medical patch is applied to and maintained on the skin of the patient. In particular, the inventive medical patch with a skin contact layer comprising a polymer II in which the saturation concentration of the active agent is negligible, and a separated active agent-containing layer comprising a polymer I in which the saturation concentration of the active agent is negligible as well practically excludes contamination with the active agent. Thus, the invention enables medical patches even comprising irritating active agents, to be touched without particular precautions, even by persons not being medical professionals, preferably by the patient itself.

According to one specific aspect, the present invention relates to a medical patch for the administration of an active agent comprising an active agent-containing self-adhesive layer structure, said active agent-containing self-adhesive layer structure comprising:
A) a backing layer;
B) an active agent-containing matrix layer comprising
   (i) an active agent in an amount of at least 0.5% by weight, based on the total weight of the active agent-containing matrix layer;
   (ii) a polymer I selected from the group consisting of silicone-based polymers and polymers based on natural or synthetic rubbers; and
   (iii) a solubilizer;
   and
C) a skin contact layer comprising a polymer II;
   wherein the skin contact layer is an adhesive layer which is directly attached to the active agent-containing layer, and
   wherein the saturation concentration of the active agent in the skin contact layer is less than 0.1% by weight.

According to certain embodiments of the invention, the medical patch according to the invention is for use in a method of treatment, in particular for use in a method of treating pain, or for use in a method of treating neuropathic pain, or nociceptive pain, or for use in a method of treating joint pain, or cancer pain, or for use in a method of treating pain associated with an articular condition, such as arthritis.

According to certain embodiments of the invention, the present invention relates to the use of a medical patch according to the invention for the manufacture of a medicament, in particular for the manufacture of a medicament for treating pain, or for the manufacture of a medicament for treating neuropathic pain, or nociceptive pain, or for the manufacture of a medicament for treating joint pain, or cancer pain, or for the manufacture of a medicament for treating pain associated with an articular condition, such as arthritis.

According to certain embodiments of the invention, the invention relates to a method of treatment, in particular a method of treating pain, or a method of treating neuropathic pain, or nociceptive pain, or a method of treating joint pain, or cancer pain, or a method of treating pain associated with an articular condition, such as arthritis, including applying a medical patch according to the invention to the skin of a patient.

Moreover, the invention relates to a method of manufacturing an active agent-containing layer structure of a medical patch according to the invention.

### Definitions

Within the meaning of this invention, the term “medical patch” refers to a dermal delivery system by which the active agent is administered to a patient, and which comprises an effective amount of the active agent in an active agent-containing (self-adhesive) structure located on a detachable protective layer (release liner). In this context, the term “medical patch” is understood to mean an adhesive patch which can be a topical medical patch or a transdermal therapeutic system (TTS). Even if the topical medical patch as well as the TTS are topically applied in the sense that they are attached to the skin of the patient, the term “topical” or “topical administration” refers to the administration of the active agent relying on passive diffusion into the skin itself, which creates a local effect at a point of action. In contrast, the term “TTS” refers to a system by which the active agent is administered to the systemic circulation via transdermal delivery.

Within the meaning of this invention, the term “active agent-containing layer structure” refers to the active agent-containing structure providing the area of release for the active agent during administration. The active agent-containing layer structure comprises at least a backing layer, an active agent-containing layer comprising the active agent, and a skin contact layer as described herein. The active agent-containing layer structure thus comprises a therapeutically effective amount of the active agent. In certain embodiments, the active agent-containing layer structure is an active agent-containing self-adhesive layer structure, thus provides adhesion to the skin so that typically no further aid for fixation on the skin is needed.

As used herein, the expression “active agent” refers to any substance of interest to be delivered by the active agent-containing layer structure to provide a beneficial or desirable effect on the condition of the subject's body either systemically or locally at the delivery site. An active agent in particular includes biologically or pharmacologically active compounds, which may also be referred to as active, drug substance, drug, active ingredient, active pharmaceutical ingredient (API), or the like. In this context, an “irritating active agent” and in particular “skin irritating active agent” is understood to mean an active agent that cause (reversible) inflammation or irritation to a body surface, including eyes, respiratory tract, skin or mucous membranes, upon contact. The degree of irritation can depend on the concentration, duration of contact, and personal factors (health status, sensitization). Considering OECD TG 404 guideline, potential (skin) irritation of medical patches can be determined, for example, by subjective in vivo observations followed by quantitation to obtain a scoring index, such as the Primary Irritation Index (PII). For quantitation, the following skin irritation scoring system can be used: Erythema and eschar formation—0 (no erythema), 1 (very slight erythema (barely perceptible)), 2 (well-defined erythema), 3 (moderate-to-severe erythema), 4 (severe erythema (beet to crimson red) to slight eschar formation (injuries in depth)); Edema formation-0 (no edema), 1 (very slight edema (barely perceptible)), 2 (slight edema (edges of area well defined by definite raising)), 3 (moderate edema (raised ~1 mm)), 4 (severe edema (raised more than 1 mm and extending beyond area of exposure)). Skin irritation is usually assumed with a PII≥0.5.

As used herein, the term “effective amount” or “therapeutically effective amount” refers to a quantity of active agent in the active agent-containing layer structure sufficient to provide, if administered by the medical patch to a patient, the desired (therapeutic) effect such as pain relief. A TTS usually contains more active agent in the system than is in fact provided to the skin and the systemic circulation, which is usually necessary to provide enough driving force for the delivery from the TTS to the systemic circulation.

Within the meaning of this invention, the terms “active”, “active agent” and the like refer to the respective active agent in any pharmaceutically acceptable chemical and morphological form and physical state. These forms include without limitation the active agent in its free base/free acid form, protonated or partially protonated form, deprotonated or partially deprotonated form, salts, cocrystals and in particular acid/base addition salts formed by addition of an inorganic or organic acid/base such as hydrochloride or tartrate salts, solvates, hydrates, clathrates, complexes and so on, as well as the active agent in the form of particles, which may be micronized, crystalline and/or amorphous, and any mixtures of the aforementioned forms.

The active agent, where contained in a medium such as a solvent, may be dissolved or dispersed or in part dissolved and in part dispersed.

When the active agent is mentioned to be used in a particular form in the manufacture of the medical patch, this does not exclude interactions between this form of the active agent and other ingredients of the active agent-containing layer structure, e.g. salt formation or complexation, in the final medical patch. This means that, even if the active agent is included in its free base/acid form, it may be present in the final medical patch in protonated or partially protonated/or deprotonated or partially deprotonated form or in the form of an acid addition salt, or, if it is included in the form of a salt, parts of it may be present as free base in the final medical patch. Unless otherwise indicated, in particular the amount of the active agent in the active agent-containing layer structure relates to the amount of the active agent included in the medical patch during manufacture of the medical patch and is calculated based on the active agent itself, but not on other forms thereof. The active agent starting material included in the medical patch during manufacture of the medical patch may be in the form of particles. The active agent may e.g. be present in the active agent-containing layer structure in the form of particles and/or dissolved.

In this context, the term “particles” refers to a solid, particulate material comprising individual particles, the dimensions of which are negligible compared to the material. In particular, the particles are solid, including plastic/deformable solids, including amorphous and crystalline materials. The term “dispersing” refers to a step or a combination of steps wherein a starting material (e.g. the active agent) is not totally dissolved. Dispersing in the sense of the invention comprises the dissolution of a part of the starting material (e.g. active agent particles), depending on the solubility of the starting material (e.g. the solubility of the active agent in the coating composition).

There are two main types of medical patches using (passive) active agent delivery, i.e. matrix-type medical patches and reservoir-type medical patches. The release of the active agent in a matrix-type medical patch is mainly controlled by the matrix including the active agent itself. In contrast thereto, a reservoir-type medical patch typically needs a rate-controlling membrane controlling the release of the active agent. In principle, also a matrix-type medical patch may contain a rate-controlling membrane. However, matrix-type medical patches are advantageous in that, compared to reservoir-type medical patches, usually no rate determining membranes are necessary and no dose dumping can occur due to membrane rupture. In summary, matrix-type medical patches are less complex in manufacture and easy and convenient to be used.

In this context, a “matrix-type medical patch” is understood to mean a system or structure wherein the active agent is homogeneously dissolved and/or dispersed within a polymeric carrier, i.e. the matrix, which forms with the active agent and optionally remaining ingredients a matrix layer. In such a system, the matrix layer controls the release of the active agent from the medical patch. Preferably, the matrix layer has sufficient cohesion to be self-supporting so that no sealing between other layers is required. Accordingly, the active agent-containing layer may be an active agent-containing matrix layer, wherein the active agent is homogeneously distributed within a polymer matrix. The active agent-containing matrix layer may comprise two active agent-containing matrix layers, which may be laminated together. Matrix-type medical patches may in particular be in the form of a “drug-in-adhesive”-type medical patch referring to a system wherein the active agent is homogeneously dissolved and/or dispersed within a pressure sensitive adhesive matrix. In this connection, the active agent-containing matrix layer may also be an active agent-containing pressure sensitive adhesive layer or active agent-containing pressure sensitive adhesive matrix layer. A medical patch comprising the active agent dissolved and/or dispersed within a polymeric gel, e.g. a hydrogel, is also considered to be of matrix-type in accordance with present invention.

Medical patches with a liquid active agent-containing reservoir are referred to by the term “reservoir-type medical patch”. In such a system, the release of the active agent is preferably controlled by a rate-controlling membrane. In particular, the reservoir is sealed between the backing layer and the rate-controlling membrane. Accordingly, the active agent-containing layer may be an active reservoir layer, which preferably comprises a liquid reservoir comprising the active agent, and wherein the active reservoir layer and the skin contact layer may be separated by the rate-controlling membrane. In the active reservoir layer, the active agent is preferably dissolved in a solvent such as ethanol or water or in silicone oil.

Reservoir-type medical patches are not to be understood as being of matrix-type within the meaning of the invention. However, microreservoir-type medical patches (biphasic systems having deposits (e.g. spheres, droplets) of an inner active agent-containing phase dispersed in an outer polymer phase), considered in the art to be a mixed form of a matrix-type medical patch and a reservoir-type medical patch that differ from a homogeneous single phase matrix-type medical patch and a reservoir-type medical patch in the concept of drug transport and drug delivery, are considered to be of matrix-type within the meaning of the present invention.

Thus, a microreservoir-type medical patch refers to a microreservoir systems, in which a liquid active agent preparation is dispersed in an adhesive matrix in the form of small droplets (“microreservoirs”). The size of the resulting droplets depends on the stirring conditions and the applied shear forces during stirring. It can be determined by an optical microscopic measurement (for example by Leica MZ16 including a camera, for example Leica DSC320) by taking pictures of the microreservoirs at different positions at an enhancement factor between 10 and 400 times, depending on the required limit of detection. By using imaging analysis software, the sizes of the microreservoirs can be determined. Microreservoirs systems are disclosed in U.S. Pat. Nos. 3,946,106, 4,053,580, 4,814,184 and 5,145,682, each of which is incorporated herein by reference. Specific microreservoirs systems are described in international patent publication WO0101967 the disclosure of which is incorporated herein by reference. These microreservoir systems contain, as base polymer, polysiloxanes and amphiphilic solvents for the microreservoir droplets.

The active agent-containing layer structure may be a pressure sensitive adhesive layer structure.

Within the meaning of this invention, the term “pressure-sensitive adhesive” (also abbreviated as “PSA”) refers to a material that in particular adheres with finger pressure, is permanently tacky, exerts a strong holding force and should be removable from smooth surfaces without leaving a residue. It is obtainable from a solvent-containing adhesive coating composition after coating on a film and evaporating the solvents (e.g. n-heptane or ethyl acetate). In this context, the term “solvent” is understood to mean any liquid substance, which preferably is a volatile organic liquid such as methanol, ethanol, isopropanol, acetone, ethyl acetate, methylene chloride, hexane, n-heptane, toluene and mixtures thereof. A pressure sensitive adhesive layer, when in contact with the skin, is self-adhesive. In certain embodiments, the active agent-containing layer structure according to the invention includes a pressure sensitive adhesive layer for skin contact which may be provided in the form of a pressure sensitive adhesive matrix or in the form of an additional layer, i.e. a pressure sensitive adhesive skin contact layer. An adhesive overlay may still be employed to advance adhesion.

Within the meaning of this invention, the term “active agent-containing layer” refers to a layer containing the active agent and providing the area of release. The term covers active agent-containing reservoir layers and active agent-containing matrix layers, and in particular active agent-containing microreservoir layers. If the active agent-containing layer is an active agent-containing matrix layer, said layer is present in a matrix-type medical patch. As used herein, the active agent-containing layer is preferably an active agent-containing matrix layer, and it is referred to the final solidified layer, e.g. obtained after coating and drying a solvent-containing coating composition as described herein. Alternatively, an active agent-containing matrix layer is obtained after melt-coating and cooling. The active agent-containing matrix layer may also be manufactured by laminating two or more such solidified layers (e.g. dried or cooled layers) of the same composition to provide the desired area weight. In certain embodiments, the matrix layer is a pressure sensitive adhesive matrix layer.

Within the meaning of this invention, the term “skin contact layer” refers to the layer included in the active agent-containing layer structure to be in direct contact with the skin of the patient during administration. The other layers of the active agent-containing layer structure do not contact the skin and do not necessarily have self-adhesive properties. The skin contact layer is directly attached to the active agent-containing layer, or a membrane is located between the active agent-containing layer and the skin contact layer. In this context, the term “membrane” is understood to mean a layer, which is provided between the active agent-containing layer and the skin contact layer and is at least semipermeable for the active agent. The membrane may be a microporous film or a nonporous partition membrane. Preferred membranes can be selected from the group consisting of polyethylene membranes, polyurethane coated polyethylene terephthalate/polyethylene membranes, polyurethane membranes, and ethylene vinyl acetate membranes. The additional skin contact layer is present as adhesive layer.

The sizes of the skin contact layer and the active agent-containing layer are usually coextensive and correspond to the area of release. However, the area of the skin contact layer may also be greater than the area of the active agent-containing layer. In such a case, the area of release still refers to the area of the active agent-containing layer.

Within the meaning of the invention, the term “backing layer” refers to a layer which supports the active agent-containing layer. At least one backing layer in the medical patch active agent-containing layer structure and usually the backing layer of the active agent-containing layer is substantially impermeable to the active agent, as well as optionally any additive, contained in the layer during the period of storage and administration and thus prevents active loss or cross-contamination in accordance with regulatory requirements. In certain embodiments, the backing layer is also occlusive, meaning substantially impermeable to water and water-vapor. Suitable materials for a backing layer include polyethylene terephthalate (PET), polyethylene (PE), ethylene vinyl acetate-copolymer (EVA), polyesters, polyurethanes, and mixtures thereof. Suitable backing layers may be siliconized in order to improve the adhesion of the active agent-containing layer to the backing layer. Alternatively, suitable backing layers may be non-siliconized in order to improve the adhesion of the active agent-containing layer to the backing layer.

Furthermore, an adhesive overlay may be present. In this context, the term “adhesive overlay” is understood to mean a layer that is free of the active agent and larger in area than the active agent-containing self-adhesive layer structure and provides additional area adhering to the skin, but no area of release of the active agent. It enhances thereby the overall adhesive properties of the medical patch. The area of said adhesive overlay adds to the overall size of the medical patch but does not add to the area of release. The adhesive overlay may comprise a self-adhesive polymer or a self-adhesive polymer mixture selected from the group of acrylic polymers, polyisobutylenes, styrene-isoprene-styrene copolymers, polysiloxanes, and mixtures thereof, which may be identical to or different from any polymer or polymer mixture included in the active agent-containing self-adhesive layer structure. The adhesive overlay comprises a backing layer that may provide occlusive or non-occlusive properties and an adhesive layer. In certain embodiments, the backing layer of the adhesive overlay provides non-occlusive properties.

Within the meaning of this invention, the term “area weight” refers to the dry weight of a specific layer, e.g. of the active agent-containing matrix layer, provided in g/m². The area weight values are subject to a tolerance of +10%, or +7.5%, due to manufacturing variability.

If not indicated otherwise “%” refers to wt. % (% by weight).

Within the meaning of this invention, the term “polymer (I or II)” refers to any substance consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2000, above 5000 or even above 10,000 Dalton. Correspondingly, compounds with a molecular weight below 2000, below 5000 or below 10,000 Dalton are usually referred to as oligomers.

Within the meaning of the invention, the term “acrylic polymer” refers to a non-hybrid polymer based on acrylates. It may be a polymer obtainable from one or more monomers selected from acrylic acid, butylacrylate, 2-ethylhexylacrylate, glycidylmethacrylate, 2-hydroxyethyl-acrylate, methylacrylate, methylmethacrylate, butylmethacrylate, t-octylacrylamide, and vinylacetate.

Within the meaning of the invention, the term “silicone-acrylic hybrid polymers” refers to a hybrid polymer based on silicones and acrylates in the form of a pressure-sensitive adhesive. Silicone acrylic hybrid pressure-sensitive adhesives are described, for example, in EP 2 599 847 and WO 2016/130408. It was found that, depending on the solvent in which the silicone acrylic hybrid PSA is supplied, the arrangement of the silicone phase and the acrylic phase providing a silicone or acrylic continuous external phase and a corresponding discontinuous internal phase is different. If the silicone acrylic hybrid PSA is supplied in n-heptane, the composition contains a continuous, silicone external phase and a discontinuous, acrylic internal phase. If the silicone acrylic hybrid PSA composition is supplied in ethyl acetate, the composition contains a continuous, acrylic external phase and a discontinuous, silicone internal phase.

Within the meaning of the invention, the term “silicone-based polymer” refers to a non-hybrid polymer (i.e. a polymer, which does not include a hybrid species) comprising polysiloxanes. Polysiloxanes can be made from solvent-free two-component systems or a solution in organic solvents. They exist in two fundamentally different variants: polysiloxanes which have free silanol groups and amine resistant polysiloxanes which are distinguished in that the free silanol groups are derivatized by trimethylsilyl groups. The methyl groups can be completely or partially replaced by other alkyl radicals or alternatively phenyl radicals. Polysiloxanes as used herein are synthesized from linear bifunctional and branched polyfunctional oligomers, the ratio of which determines the physical properties thereof. More polyfunctional oligomers result in a more cross-linked adhesive with a higher cohesion and a reduced tack, less polyfunctional oligomers result in a higher tack and a reduced cohesion. It is preferred for the silicone-based polymer to be a mixture of high tack and medium tack, or high tack and low tack, polysiloxanes. In certain embodiments, the at least one silicone-based polymer is a silicone-based pressure sensitive adhesive.

Within the meaning of this invention, the term “silicone gel adhesive” refers to an elastic, jelly-like material formed by lightly crosslinking silicone polymers. It may be prepared from a gel producing composition as described further below upon curing. In particular, the silicone gel adhesive forms upon curing of polysiloxanes comprising reactive groups such as Si—H reactive groups and aliphatic unsaturated groups, which react with each other in the presence of a hydrosilylation catalyst. In certain embodiments, the silicone gel adhesive is based on a polydimethylsiloxane network, which may be formed in an addition reaction (hydrosilylation) between vinyl function polydimethylsiloxane groups (polymer) and hydrogen functional siloxanes (cross-linker). Accordingly, the silicone gel adhesive is typically applied by using a curable gel producing (2-component) composition, which solidifies upon curing.

Within the meaning of the invention, the term “natural or synthetic rubbers” refers to an elastomer which is obtainable by polymerizing an unsaturated hydrocarbon, such as isoprene (2-methyl-1,3-butadiene), or by copolymerizing such hydrocarbons with styrene, butadiene, or the like. It includes natural and synthetic polyisoprene, polybutylene and polyisobutylene, styrene/butadiene polymers, styrene-isoprene-styrene block copolymers, hydrocarbon polymers such as butyl rubber, halogen-containing polymers such as polyacrylic-nitrile, polytetrafluoroethylene, polyvinylchloride, polyvinylidene chloride, and polychlorodiene, as well as other copolymers thereof. In certain embodiments, natural or synthetic rubbers may be styrenic triblock copolymers or polyisobutylenes.

Within the meaning of this invention, the term “saturation concentration” refers to that active agent concentration corresponding to the equilibrium state in which the solvent (i.e. the polymer I of the active agent-containing layer or the polymer II of the skin contact layer) cannot dissolve further solute (i.e. the active agent), and as a result the solid solute is present in equilibrium with the solid solution at defined temperature (room temperature-unmodified temperature found indoors in the laboratory where experiments are conducted and usually lies within 15 to 35° C., or about 18 to 25° C.). The saturation concentration of the active agent can be indicated in % by weight, based on the total weight of the active agent layer or skin contact layer, respectively. The saturation concentration can be determined e.g. using a method described by Liu, P., Gargiulo, P., Wong, J., and Novartis. Pharm. Research. Vol. 14, p. 317 (1997), herein referred to as “Sandwich method”, in which a multi-layered laminate is prepared comprising an upper and lower protective layer sandwiching a donor layer and an acceptor layer separated by a partitioning membrane that is permeable to the active agent. Since the donor layer contains an excess of the active agent and the acceptor layer is substantially free of the active agent, the active agent diffuses out of the donor layer through the partitioning membrane into the acceptor layer until the saturation concentration is achieved. The donor layer and the acceptor layer are manufactured from the respective polymer II of the skin contact layer (or the respective polymer I of the active agent-containing layer). The donor layer is oversaturated with the active agent, while the acceptor layer is prepared analogously to the donor layer but does not comprise the active agent. The sandwich systems prepared are stored for a certain time, e.g., 7 days, at room temperature, to allow the active agent to diffuse from the donor layer into the acceptor layer. Then, the remaining active agent concentration of the donor layer is determined by means of HPLC (high performance liquid chromatography) in order to finally obtain the saturation concentration of the active agent in the respective polymer II of the skin contact layer (or the respective polymer I of the active agent-containing layer).

As used herein, solubility parameters (SPs) are defined as the sum of all the intermolecular attractive forces, which, as a numerical estimate, are empirically related to the extent of mutual solubility of chemical species. The most convenient method to determine solubility parameters is Hildebrand's method, which computes the solubility parameter from molecular weight, boiling point and density data, which are commonly available for many materials: SP=(ΔEᵥ/V)^{1/2}, where V=molecular weight/density and ΔEᵥ=energy of vaporization. For materials, such as high molecular weight polymers, which have vapor pressures too low to detect, several methods have been developed which use the summation of atomic and group contributions to vaporization. Such a method of calculating the solubility parameter of a material has been described e.g. by Small, J. Applied Chem. Vol. 3, p. 71 (1953). Some solubility parameters (calculated by Small's method) of exemplary polymers useful in the practice of the invention are as follows: Polydimethylsiloxane 14.9 MPa^{1/2}, polyisobutylene 15.7 MPa^{1/2}, polyethylene/butylene 16.2 MPa^{1/2}, polyisoprene 16.6 MPa^{1/2}, polyethylene 16.6 MPa^{1/2}, polybutadiene 16.6 MPa^{1/2}, polybutadiene-co-styrene (75/25 to 72/28) 17.4 MPa^{1/2}, polystyrene 18.6 MPa^{1/2}, polymethyl methacrylate 19.0 MPa^{1/2}, polymethyl acrylate 19.8 MPa^{1/2}.

Within the meaning of this invention, the term “solubilizer” refers to an agent that increases the solubility of the active agent in the active agent-containing matrix layer.

It should be noted that in pharmaceutical formulations, the formulation components are categorized according to their physicochemical and physiological properties, and in accordance with their function. This means in particular that a substance or a compound falling into one category is not excluded from falling into another category of formulation component. The skilled person is able to determine based on his general knowledge in which category or categories of formulation component a certain substance or compound belongs to. For example, the solubilizer as defined above may also act as e.g., permeation enhancer. On the other hand, a permeation enhancer as used within the meaning of this invention does not necessarily increases the solubility of the active agent in the active agent-containing layer.

Within the meaning of the invention, the term “soluble polyvinylpyrrolidone” refers to polyvinylpyrrolidone, also known as povidone, which is soluble with more than 10% in at least ethanol, preferably also in water, diethylene glycol, methanol, n-propanol, 2 propanol, n-butanol, chloroform, methylene chloride, 2-pyrrolidone, macrogol 400, 1,2 propylene glycol, 1,4 butanediol, glycerol, triethanolamine, propionic acid and acetic acid. Examples of polyvinylpyrrolidones which are commercially available include Kollidon® 12 PF, Kollidon® 17 PF, Kollidon® 25, Kollidon® 30 and Kollidon® 90 F supplied by BASF, or povidone K90F. The different grades of Kollidon® are defined in terms of the K-Value reflecting the average molecular weight of the polyvinyl-pyrrolidone grades. Kollidon® 12 PF is characterized by a K-Value range of 10.2 to 13.8, corresponding to a nominal K-Value of 12. Kollidon® 17 PF is characterized by a K-Value range of 15.3 to 18.4, corresponding to a nominal K-Value of 17. Kollidon® 25 is characterized by a K-Value range of 22.5 to 27.0, corresponding to a nominal K-Value of 25, Kollidon® 30 is characterized by a K-Value range of 27.0 to 32.4, corresponding to a nominal K-Value of 30. Kollidon® 90 F is characterized by a K-Value range of 81.0 to 97.2, corresponding to a nominal K-Value of 90. Preferred Kollidon® grades are Kollidon® 12 PF, Kollidon® 30 and Kollidon® 90 F. In this contex, the term “K-Value” refers to a value calculated from the relative viscosity of polyvinylpyrrolidone in water according to the European Pharmacopoeia (Ph.Eur.) and USP monographs for “Povidone”. For all grades and types of polyvinylpyrrolidone, it is preferred that the amount of peroxides is within certain limits, in particular, the peroxide amount is equal to or less than 500 ppm, equal to or less than 150 ppm, or equal to or less than 100 ppm.

Within the meaning of the invention, the term “hexagonal shape” refers to the two-dimensional shape of the active agent-containing layer structure which is provided by the backing layer and the active agent-containing layer, or the backing layer, the active agent-containing layer and the skin contact layer, respectively, and which can be seen when regarding the active agent-containing layer structure from above onto the backing layer. A hexagonal shape in the sense of the present invention is understood as any shape that can be formed by one hexagon or by an aggregation of two or more hexagons. This means, the hexagonal shape according to the invention as a whole does not need to have the shape of a hexagon, but is to be constituted by at least one hexagon. The vertexes of the hexagonal shape may be sharp or rounded. If two or more hexagons constitute the hexagonal shape, they may be integrally connected to each other, i.e. only separable by e.g. cutting the active agent-containing layer structure, or may be detachably connected to each other using e.g. a perforated line. The backing layer and the active agent-containing layer, or the backing layer, the active agent-containing layer and the skin contact layer, respectively, as well as optionally the membrane, are coextensive, i.e. they have the same planar extent and/or share the same boundary. In other words, the backing layer and the active agent-containing layer, or the backing layer, the active agent-containing layer and the skin contact layer, respectively, as well as optionally the membrane, each provides a shape in the form of at least one congruent hexagon.

Within the meaning of the invention, the term “hexagon” refers to a six-sided polygon. In a “convex hexagon”, each of the six points where two sides of the hexagon meet in pairs (vertices) points outwards. Two adjacent vertices are respectively connected by one of the six sides (common side). Non-adjacent vertices can be connected by one of nine diagonals lying inside the closed hexagonal chain (boundary) of the convex hexagon. A convex hexagon requires the least total length of the boundary, compared to other polygons having the same area.

A convex hexagon may also be described as a six-sided polygon having interior angles (vertex angles) which are each less than 180°. The total of the interior angles of any simple (non-self-intersecting) hexagon is 720°. Thus, a (convex) hexagon whose vertex angles are equal to 120° each is also referred to as being equiangular. A (convex) hexagon having all sides of equal length is also referred to as being equilateral. If a (convex) hexagon is both equilateral and equiangular, it is also referred to as being regular.

The (convex) hexagon may be symmetrical, in particular mirror symmetrical or rotational symmetrical. In this context, mirror symmetry, also referred to as reflection symmetry, is understood to mean symmetry with respect to a reflection. Such symmetric function of a two-dimensional shape is that if the shape were to be folded half over the mirror axis, the two halves would be identical: the two halves are each other's mirror images. Thus, a regular hexagon has six axes of symmetry, because there are six different ways to fold it and have the sides all match. Rotational symmetry of order n, also called n-fold rotational symmetry, of a two-dimensional shape with respect to a particular point is understood to mean that rotation by an angle of 360°/n (180°, 120°, 90°, 72°, 60°, etc.) does not change the shape. Thus, a regular hexagon has rotational symmetry of order 6, because it looks the same after each of a partial turn about an angle of 60°.

Within the meaning of the invention, the term “parallelogon” refers to a (convex) hexagon wherein all pairs of opposite sides (two sides being separated from each other by the same number of sides in both boundary directions, i.e., by two sides in a hexagon) are parallel and the two sides of each pair of parallel opposite sides are equal in length. This includes a parallelogon having in total three different side length or having in total only two different side length, as well as a parallelogon having all sides of equal length. The term “parallelogon” includes, e.g., a shape obtained by elongating a parallelogram or a shape obtained by elongating a rhombus, and in particular by separating a parallelogram or rhombus, respectively, at two of its non-adjacent vertices and introducing a pair of parallel opposite sides of equal length. In this context, the term “parallelogram” is understood to mean a simple four-sided polygon with two pairs of parallel sides. If the four sides are of equal length, the parallelogram is also referred to as “rhombus”.

Within the meaning of the invention, the term “aspect ratio” refers to the height-to width ratio wherein the width and length are distances between two points on the boundary of the (convex) hexagon and the longer of the two distances is regarded as the width. In this context, the width of the (convex) hexagon is understood to mean the length of the longest distance between any two points on the boundary, which is often the length of the longest diagonal of the (convex) hexagon between two diametrically opposite vertices. The height of the (convex) hexagon is understood to mean the longest distance available between any two points on the boundary of the (convex) hexagon so that the line formed by connecting these two points is perpendicular to the line formed by connecting the two points defining the width (see above). The height-to-width-ratio corresponds to the ratio of inradius (radius of the inscribed circle) to circumradius (radius of the circumscribed circle), if available. The height-to width ratio of a regular hexagon is √3:2.

Regular hexagons fit together to tile the plane forming a honeycomb pattern. The honeycomb pattern is composed of regular hexagons arranged side by side, which tile the plan, i.e., completely fill the entire surface they span, so there are not any holes in between them. This is because the 120° angle is the angle at which the sides meet at the vertices when the hexagons are lined side by side, such that exactly three hexagons meeting at every vertex. The honeycomb pattern appears not only in honeycombs but also in many other places in nature, such as, e.g., in organic compounds (benzyl rings, proteins).

Hexagonal tiling, also referred to as hexagonal tessellation, is a regular tiling of the Euclidian plane, in which exactly three hexagons meet at each vertex. Besides using regular hexagons (FIG. 1a), hexagonal tiling may also be carried out with, for example, other (hexagonal) parallelogons, in particular with a parallelogon obtained by elongating a rhombus (FIG. 1b) or obtained by elongating a parallelogram (FIG. 1c). Such hexagonal shapes can tile the Euclidean plane by translation. Other hexagon shapes can tile the plane with different orientations. In this context, the term “tile the plane” is understood to mean the coverage of a particular plane (plane surface or curved surface) without leaving any gaps. Tiling the plane may be carried out with or without overlapping adjacent active agent-containing layer structures according to the present invention. Preferably, overlapping is avoided as much as possible.

Within the meaning of the invention, the term “pentagonal shape” refers to the two-dimensional shape of the active agent-containing layer structure which is provided by the backing layer and the active agent-containing layer, or the backing layer, the active agent-containing layer and the skin contact layer, respectively, and which can be seen when regarding the active agent-containing layer structure from above onto the backing layer. A pentagonal shape in the sense of the present invention is understood as any shape that can be formed by one pentagon or by an aggregation of two or more pentagons. This means, the pentagonal shape according to the invention as a whole does not need to have the shape of a pentagon, but is to be constituted by at least one pentagon. The vertexes of the pentagonal shape may be sharp or rounded. If two or more pentagons constitute the pentagonal shape, they may be integrally connected to each other, i.e. only separable by e.g. cutting the active agent-containing layer structure, or may be detachably connected to each other using e.g. a perforated line. The backing layer and the active agent-containing layer, or the backing layer, the active agent-containing layer and the skin contact layer, respectively, as well as optionally the membrane, are coextensive, i.e. they have the same planar extent and/or share the same boundary. In other words, the backing layer and the active agent-containing layer, or the backing layer, the active agent-containing layer and the skin contact layer, respectively, as well as optionally the membrane, each provides a shape in the form of at least one congruent pentagon.

Within the meaning of the invention, the term “pentagon” refers to a five-sided polygon. In a “convex pentagon”, each of the five points where two sides of the pentagon meet in pairs (vertices) points outwards. One vertex is respectively formed by two of the five sides (adjacent sides). Two adjacent vertices are respectively connected by one of the five sides (common side). Non-adjacent vertices can be connected by one of five diagonals lying inside the closed pentagonal chain (boundary) of the convex pentagon.

A convex pentagon may also be described as a five-sided polygon having interior angles (vertex angles) which are each less than 180°. The total of the interior angles of any simple (non-self-intersecting) pentagon is 540°. Thus, a (convex) pentagon whose vertex angles are equal to 108° each is also referred to as being equiangular. A (convex) pentagon having all sides of equal length is also referred to as being equilateral. If a (convex) pentagon is both equilateral and equiangular, it is also referred to as being regular. If a (convex) pentagon has at least one of the five vertex angles of other size and/or at least one of the five sides of other length, it is herein referred to as being non-regular.

The (convex) pentagon may be symmetrical, in particular mirror symmetrical. In this context, mirror symmetry, also referred to as reflection symmetry, is understood to mean symmetry with respect to a reflection. Such symmetric function of a two-dimensional shape is that if the shape were to be folded half over the mirror axis, the two halves would be identical: the two halves are each other's mirror images. Preferred (convex) pentagons according to the present invention have at least one, in particular exactly one axis of symmetry.

With the meaning of the invention, the term “pentagon of type I” as well as “pentagon of type II” refer to a mirror symmetrical convex pentagon with exactly one axis of symmetry, having two interior angles of 90° and three interior angles of 120°, one of which being divided by the axis of the symmetry in the middle. Further, the “pentagon of type I” and the “pentagon of type II” both have one pair of sides of equal length and one other pair of sides of equal length (which may be the same or a different length), as well as one remaining side that is divided by the axis of symmetry in the middle (the remaining side having the same or different length as/than the one pair of sides of equal length and/or the other pair of sides of equal length). Finally, the “pentagon of type I” differs from the “pentagon of type II” in that the two interior angles of 90° are adjacent, thus one pair of sides of equal length is parallel (the sides being parallel to one another as well as to the axis of symmetry).

Non-regular pentagons having two interior angles that sum up to 180° fit together to tile the plane. There are 15 monohedral convex pentagonal tilings known, the most recent one of which was discovered in 2015 (FIG. 1; column 1, 2 and 3, from top to bottom):

$$Type 1 : B + C = 180 ° , A + D + E = 360 °$$

$$Type 2 : B + D = 180 ° , c = e$$

$$Type 3 : A = C = D = 120 ° , a = b , d = c + e$$

$$Type 4 : B = D = 90 ° , b = c , d = e$$

$$Type 5 : A = 60 ° , D = 120 ° , a = b , d = e$$

$$Type 6 : B + D = 180 ° , 2 B = E , a = d = e , b = c$$

$$Type 7 : B + 2 E = 2 C + D = 360 ° , b = c = d = e$$

$$Type 8 : 2 B + C = D + 2 E = 360 ° , b = c = d = e$$

$$Type 9 : 2 A + C = D + 2 E = 360 ° , b = c = d = e$$

$$Type 10 : A = 90 ° , B + E = 180 ° , B + 2 C = 360 ° , a = b = c + e$$

$$Type 11 : A = 90 ° , C + E = 180 ° , 2 B + C = 360 ° , 2 a + c = d = e$$

$$Type 12 : A = 90 ° , C + E = 180 ° , 2 B + C = 360 ° , 2 a = d = c + e$$

$$Type 13 : B = E = 90 ° , 2 A + D = 360 ° , d = 2 a = 2 e$$

$$Type 14 : A = 90 ° , B \approx 145.34 ° , C \approx 69.32 ° , D \approx 124.66 ° , E \approx 110.68 ° ,$$

$$2 a = 2 c = d = e$$

$$Type 15 : A = 150 ° , B = 60 ° , C = 135 ° , D = 105 ° , E = 90 ° , a = c = e ,$$

$$b = 2 a$$

This list has shown to be complete by Rao in 2017 (Rao, Michaël: “Exhaustive search of convex pentagons which tile the plane.”).

Pentagonal tiling, also referred to as pentagonal tessellation, is a tiling of the plane, in which each individual piece is in the shape of a pentagon. Regular pentagons do not tile the Euclidian plane, however, they can tile a sphere with three pentagons and the hyperbolic plane with four or more pentagons around each vertex. The above types of pentagons that can monohedrally tile the Euclidian plane (i.e. with one type of tile) not have any symmetry in general, although some have special cases with mirror symmetry. For example, the pentagon of type I of the present invention is a mirror symmetrical case of Type 1, and the pentagon of type II is a mirror symmetrical case of Type 3 or Type 4. A pentagon of type I, in which the sides of the pair of parallel sides of equal length as well as the remaining side have a length that is √3 times the length of the sides of the other pair of sides of equal length can tile the Euclidian plane in prismatic pentagonal tiling (FIG. 2a). A pentagon of type II, in which the remaining side has a length that is √3-1 times the length of the sides of the one pair of sides of equal length and of the other pair of sides of equal length can tile the Euclidian plane in Cairo pentagonal tiling (FIG. 2b).

In addition, pentagons have a peculiar relationship with hexagons as some types of hexagons can be subdivided into pentagons. For example, a convex hexagon can be subdivided into two Type 1 pentagons, three or nine Type 3 pentagons, or four Type 4 pentagons. Thus, a pentagon of type I, in which the sides of the pair of parallel sides of equal length have a length that is half the length of the sides of the other pair of sides of equal length, and the remaining side has a length that is √3 times the length of the sides of the other pair of sides of equal length can provide a monohedral pentagonal tiling with overlays of regular hexagons each comprising two pentagons. Also, a pentagon of type II, in which the sides of the one pair of sides of equal length have a length that is half the length of the remaining side, and the sides of the one other pair of sides of equal length have a length that is 2/√3 times the length of the remaining side can provide a monohedral pentagonal tiling with overlays of regular hexagons each comprising three pentagons.

Within the meaning of the invention, the term “sheet of medical patches” refers to a number of medical patches sharing a common release liner. Each of the medical patches represents an individual dosing unit that may be applied to the skin of the patient after peel-off from the release liner. The amount of active agent contained in the medical patch refers to the amount of active agent contained in the active agent-containing layer structure of the medical patch. The amount of active agent contained in the sheet of medical patches refers to the total amount of active agent contained in all active agent-containing layer structures of the medical patches constituting the sheet of medical patches. Accordingly, the area of release of the medical patch refers to the area provided by the active agent-containing layer structure of the medical patch, and the area of release of the sheet of medical patches refers to the area provided by all active agent-containing layer structures of the medical patches constituting the sheet of medical patches.

Within the meaning of the invention, the term “release liner” refers to a detachable protective layer, attached to the active agent-containing layer or the skin contact layer of the active agent-containing layer structure(s). The release liner may have any suitable two-dimensional geometric shape and preferably has a polygonal shape, in particular a rectangular or square shape. Preferably, the area of the release liner encompasses the total area of all active agent-containing layer structures of the medical patches constituting the sheet of medical patches. The release liner may be coextensive with the active agent-containing layer structures or extends beyond the boundary formed by all active agent-containing layer structures in all directions, i.e. the polygonal chain formed by the outside sides of the hexagons and/or pentagons of the active agent-containing layer structures completely lies within or on the polygonal chain formed by the sides of the release liner. Suitable release liners may be polyethylene terephthalate (PET) or polypropylene (PP) films, optionally provided with a silicone or fluoropolymer coating. This included, e.g., commercially available release liners, such as the Scotchpak® release liners 9741/9742/9744 of 3M.

Within the meaning of the invention, the term “weakened” refers to the result of any action (weakening) enabling easier separation of two sections of an active agent-containing layer structure or of two different active agent-containing layer structures, although the two sections/active agent-containing layer structures are still connected to each other. Such weakening may include but is not limited to folding, scratching, perforating, piercing, puncturing, punching, or cutting. In certain embodiments, the weakening is carried out by perforation. In this context, the term “perforated” is understood to mean having small holes made in it. Perforation may be obtained by, e.g., needling or laser cutting.

Within the meaning of the invention, the term “fastening bridge” refers to a single point between two or three active agent-containing layer structures, in particular between two or three hexagonal and/or pentagonal shapes, at which they are still connected, while the main part of the common sides is cut or weakened. It is preferably obtained by leaving the connection during the separation process, which may be carried out by, e.g. punching or cutting. The fastening bridge(s) enable(s) joint peel-off of active agent-containing layer structures connected in this manner from the release liner. In addition, the fastening bridge(s) is/are preferably so thin that they can easily be undone, e.g. by pulling on a part of the active agent-containing layer structures, in order to separate some of the active agent-containing layer structures from the others.

Within the meaning of this invention, the term “patient” refers to a subject who has presented a clinical manifestation of a particular symptom or symptoms suggesting the need for treatment, who is treated preventatively or prophylactically for a condition, or who has been diagnosed with a condition to be treated. Preferably, the patient suffers from neuropathic pain, or nociceptive pain, or mixed neuropathic and nociceptive pain such as joint pain or cancer pain.

Within the meaning of this invention, the term “neuropathic pain” refers to pain caused by a lesion or disease of the somatosensory nervous system. In this context, the term “chronic neuropathic pain” is understood to mean neuropathic pain lasting for at least three months. When suffering from neuropathic pain, most patients complain of an ongoing or intermittent spontaneous pain of, e.g., burning, pricking, squeezing quality, which may be accompanied by evoked pain, particular to light touch and cold. Ectopic activity in, e.g., nerve-end neuroma, compressed nerves or nerve roots, dorsal root ganglia, and the thalamus may in different conditions underlie the spontaneous pain. Neuropathic pain includes peripheral neuropathic pain that particularly affects the peripheral nerves, meaning the nerves located outside the brain and spinal cord. In particular, neuropathic pain within the meaning of this invention relates to post-surgical neuropathic pain, as well as neuropathic pain associated with postherpetic neuralgia or diabetic peripheral neuropathy of the hands or feet.

In this context, the term “post-surgical neuropathic pain” is understood to mean chronic pain that develops after a surgical procedure and persists beyond the healing process, i.e. at least three months after the surgery. The pain is either localized to the surgical field or area of injury, projected to the innervation territory of a nerve situated in this area, or referred to a dermatome (after surgery/injury to deep somatic or visceral tissues). Chronic post-surgical pain is the result of nerve damage and can be due to the surgery itself or other causes of pain including infection, malignancy, etc.

In this context, the term “postherpetic neuralgia”, also referred to as post-shingles nerve pain, is understood to mean pain occurring if nerves are damaged due to a previous herpes zoster infection, commonly referred to as shingles. Symptoms of post-shingles nerve pain are often limited or localized to the area of the skin where the shingles outbreak first occurred, in the band around the trunk, usually on one side of the body. Less common symptoms of post-shingles nerve pain include itching, numbness, or feeling “pins and needles.”

In this context, the term “diabetic peripheral neuropathy”, also referred to as diabetic nerve pain”, is understood to mean pain occurring if nerves are damaged as a result of diabetes. Although diabetic nerve pain can affect any nerve, it is most often felt in extremities, such as the hands or feet.

Within the meaning of the invention, the term “nociceptive pain” refers to pain that is caused by structural dysfunctions such as damage to body tissue. It is the normal response to noxious (intense) stimulation, predictably initiated by the activation of nociceptors, i.e. primary afferent neurons with a high activation threshold that detect signals from damaged tissues or the threat of damage. Nociceptive pain can range from sharp, pricking, or shock-like to dull, aching, or burning depending on the responsible stimulus, and continues only as long as the noxious stimulus is maintained. That said, sustained or recurrent noxious stimulation can occur in certain disease states like osteoarthritis, where changes in the joint can allow normal weight bearing to produce forces sufficient to activate nociceptors. Nociceptive pain may be subdivided into somatic pain and visceral pain.

In this context, the term “somatic pain” is understood to mean nociceptive pain originating from nociceptors in the skin, bones, connective tissue, muscles, or joints. Somatic pain is characterized as well localized, intermittent, or constant and described as aching, gnawing, throbbing, or cramping. It may progress into a dull ache with time, particularly for deep somatic pain in the muscles and bones.

In this context, the term “visceral pain” is understood to mean nociceptive pain originating from nociceptors in or near the internal organs. In particular, visceral pain is mediated by discrete nociceptors in the cardiovascular, respiratory, gastrointestinal, and genitourinary systems and is usually described as deep, squeezing, or colicky, and is commonly referred to cutaneous sites, which may be tender.

Within the meaning of this invention, the term “joint pain”, also referred to as arthralgia, refers to an articular condition, such as discomfort, aches or soreness in any of the body's joints of a patient, including spine, shoulders, hips, elbows, knees, feet and finger joints. In this context, joints are understood to be pivotal structures of the articular system that can be considered as discontinuities in the skeleton permitting controlled mobility, and can have different structures depending on their functional requirements. Joint pain may be constant or come and go. It may be a result of illness or injury but can also be due to other conditions or factors. In particular, joint pain tends to affect people who have arthritis or other long-term (chronical) medical conditions.

Within the meaning of this invention the term “articular condition” refers to any disease, disorder or discomfort affecting or involving a joint or joints. The articular condition may be inflammatory, i.e. involve inflammation of the bones or related tissues. It may include angiogenesis, i.e. the growth of new capillary blood vessels from pre-existing vasculature, and/or arthropathy, i.e. a particular abnormal condition that negatively affects the structure or function of all or part of the joint(s), in particular arthritis.

In this context, the term “arthritis” is understood to mean a form of arthropathy that involves inflammation of one or more joints. The arthritis may be infectious, i.e. caused by bacterial, viral or fungal infection that spreads from another part of the body, or non-infectious, i.e. caused by other factors. It may include rheumatoid arthritis, i.e. a long-term autoimmune disorder that primarily affects joints, juvenile arthritis, i.e. an autoimmune, non-infective, inflammatory joint disease that is onset before 16 years of age, psoriatic arthritis, a long-term inflammatory arthritis caused by psoriasis, gouty or pseudogouty arthritis, i.e. a form of inflammatory arthritis caused by needle-like crystals of uric acid know as monosodium urate crystals or caused by calcium pyrophosphate dehydrate crystals, respectively, or osteoarthritis, i.e. a degenerative disease characterized by cartilage erosion, bony hypertrophy, subchondral sclerosis, and synovial and capsular changes. The articular condition may result in particular in knee pain, elbow pain, hip pain, shoulder pain, pain of the hands or feet, or pain of the (lower) back, which are preferably nociceptive at first but may also comprise neuropathic elements.

Within the meaning of this invention, the term “cancer pain” relates to neuropathic cancer pain caused by nerve damage attributable to the cancer per se, and/or treatments including chemotherapy, radiotherapy, and surgery. Cancer pain caused by the tumor per se usually involves both nociceptive and neuropathic components, and mixed pain is more common than neuropathic cancer pain caused by cancer treatments. Most cancer pain caused by chemotherapy is purely neuropathic in nature. Neuropathic cancer pain is nerve-related (typically neuron-related) pain characterized as a burning or electrical sensation; however, it sometimes manifests as decreased sensation or actual muscle weakness.

According to the invention, the medical patch as described herein is suitable for use in a method of treatment, preferably in which the medical patch is applied for a short period of time (application time), but provides for the desired effect of pain relief for an extended period of time (effect time). Within the application time, it is preferred for the medical patch to release almost the total amount of the active agent contained in the active agent-containing layer of the medical patch. In certain embodiments, the active agent is able to desensitize and defunctionalize a corresponding pain receptor within the application time, so that it can provide sustained pain relief that lasts during the effect time.

Within the meaning of this invention, the term “short period of time”, i.e., the application time, relates to a period of less than or about 240 minutes, less than or about 180 minutes, less than or about 120 minutes, less than or about 90 minutes, less than or about 60 minutes, less than or about 45 minutes, less than or about 30 minutes, less than or about 15 minutes, or 30 to 90 minutes.

Within the meaning of this invention, the term “extended period of time”, i.e., the effect time. relates to a period of at least or about 1 week, at least or about 2 weeks, at least or about 1 month, at least or about 1.5 months, at least or about 2 months, at least or about 3 month, or 1 to 3 months.

The interval between two dosage form administrations, also called dosing interval, needs to be adapted accordingly. Within the meaning of the present invention, the term “dosing interval” refers to the period of time between two consecutive medical patch administrations, i.e. the interval between two consecutive points in time a medical patch is applied to the skin of the patient. Once applied, the medical patch is only maintained on the skin of the patient for the application time and subsequently removed. However, the dosing interval lasts until a new medical patch is applied to the skin.

As used herein, the term “medical professional” is understood to mean a provider of health care treatment and advice based on formal training and experience. This is in particular a person being trained in application and removal of medical patches (carrying irritating active agents).

Within the meaning of this invention, the term “coating composition” refers to a composition comprising all components of the active agent-containing layer or the skin contact layer, respectively, which may be coated onto an intermediate liner, the backing layer or release liner to form the active agent-containing layer and the skin contact layer upon drying (or curing).

Within the meaning of this invention, the term “dissolve” refers to the process of obtaining a solution, which is clear and does not contain any particles, as visible to the naked eye.

Within the meaning of this invention, the term “cross-linking” refers to the process of cross-link functional groups that may be contained within the active-free coating composition.

Within the meaning of this invention, and unless otherwise specified, the term “about” refers to an amount that is ±10% of the disclosed amount. In some embodiments, the term “about” refers to an amount that is ±5% of the disclosed amount. In some embodiments, the term “about” refers to an amount that is ±2% of the disclosed amount.

### BRIEF DESCRIPTION OF THE DRAWINGS

FIG. 1a depicts the cumulative permeated amount of medical patches prepared according to Example 1.

FIG. 1b depicts the skin permeation rate of medical patches prepared according to Example 1.

### DETAILED DESCRIPTION

### Medical Patch Structure

The present invention relates to a medical patch for the administration of an active agent. The medical patch can be either a topical medical patch or a transdermal therapeutic system. In certain embodiments, the patch is a topical medical patch, in particular for the topical administration of the active agent.

The medical patch according to the present invention comprises an active agent-containing layer structure, said active agent-containing layer structure comprising:
A) a backing layer;
B) an active agent-containing layer comprising
   (i) an active agent, and
   (ii) a polymer I;
   and
C) a skin contact layer comprising a polymer II;
   wherein the skin contact layer is an adhesive layer which is directly attached to the active agent-containing layer.

Preferably, the aforementioned layers of the medical patch according to the invention are directly attached to each other, i.e. the backing layer is directly attached to the active agent-containing layer, which is on the other side directly attached to the skin contact layer. In other words, the medical patch according to the present invention comprises its layers in the following order: (1) backing layer, (2) active agent-containing layer, and (3) skin contact layer.

The backing layer, the active agent-containing layer, and the skin contact layer may be coextensive, i.e. they have the same planar extent (size). Alternatively, it might be preferred for the backing layer and/or the skin contact layer to be larger in area than the active agent-containing layer. With an active agent-containing layer structure construed like this, the active agent contained in the active agent-containing layer is even more safely enclosed therein.

The additional skin contact layer preferably provides for adhesion between the active agent-containing layer structure and the skin of the patient during administration. In some embodiments, the active agent-containing layer structure is a self-adhesive layer structure. The active agent-containing layer structure according to the present invention, which comprises an additional skin contact layer, when applied to a patient's skin, provides improved wearing properties as well as a clean and painless removal. If necessary, e.g. in the case of repositioning, the medical patch can be removed and applied again without loss of the adhesiveness. In addition, the structure of the active agent-containing layer structure sandwiching the active agent-containing layer between the skin contact layer (in which the active agent is preferably substantially insoluble) and the backing layer (which is preferably substantially impermeable to the active agent) shields the active agent from the skin of the patient or other applying/removing person before and/or after application of the medical patch.

The medical patch according to the present invention may be a matrix-type medical patch or a reservoir-type medical patch, and preferably is a matrix-type medical patch. In certain embodiments, the medical patch according to the present invention is a matrix-type medical patch, wherein the active agent is homogeneously dissolved and/or dispersed within a polymeric carrier, i.e. the matrix, which forms together with the active agent and optionally further additives a matrix layer. In certain embodiments, the active agent-containing matrix layer may comprise at least the active agent and a solubilizer dissolved and/or dispersed within the matrix (i.e. the polymer I). Thus, the medical patch according to the invention may be a microreservoir-type medical patch.

The active agent-containing layer structure according to the invention is normally located on a detachable protective layer (release liner) from which it is removed immediately before application to the surface of the patient's skin. Thus, according to certain embodiments, the medical may further comprise a release liner. A medical patch protected this way is usually stored in a seam-sealed pouch. The packaging may be child resistant and/or senior friendly.

### Active Agent-Containing Layer

As outlined in more detail above, the medical patch according to the present invention comprises an active agent-containing layer structure comprising inter alia an active agent-containing layer, which comprises
(i) an active agent, and
(ii) a polymer I.

In accordance with the invention, the active agent-containing layer is sandwiched between the skin contact layer and the backing layer, thus not being allowed to come into contact with a patient's (or other applying/removing person's) skin. Accordingly, the layers of the active agent-containing layer structure are selected so as the active agent-containing layer is the only layer permitting the active agent to concentrate to more than 0.1% by weight (i.e. neither the skin contact layer nor the backing layer should be able to carry the active agent in an amount of more than 0.1% by weight.)

Thus, in certain embodiments, the saturation concentration of the active agent in the active agent-containing layer is more than 1% by weight, more than 5% by weight, more than 10% by weight, or more than 20% by weight. In particular, the saturation concentration of the active agent in the polymer I of the active agent-containing layer may be more than 1% by weight, more than 5% by weight, more than 10% by weight, or more than 20% by weight. Alternatively, the saturation concentration of the active agent in the polymer I of the active agent-containing layer may be (effectively) less than 1% by weight, or less than 0.1% by weight, and may only be locally increased by the addition of one or more solubilizer(s).

Thus, in particular embodiments, the active agent-containing layer may further comprise a solubilizer, in particular in an amount of from 1 to 40% by weight, based on the total weight of the active agent-containing layer. Accordingly, the active agent-containing layer may comprise
(i) an active agent,
(ii) a polymer I, and
(iii) a solubilizer.

Without wishing to be bound by theory, it is believed that the safe and uncomplicated handling of the medical patch according to the present invention, relying on the skin contact layer providing a saturation concentration of the active agent of less than 0.1% by weight, can be further improved by selecting the polymer I of the active agent-containing layer so as to provide a saturation concentration of the active agent of less than 1% by weight, or less than 0.1% by weight, and adding a solubilizer forming deposits, e.g., droplets with the active agent in a microreservoir system. By increasing the active agent concentration in the active agent-containing layer locally only, the active agent can be maintained within the active agent-containing layer as long as the active agent-containing layer structure is not in contact to the patient's skin. Thus, unwanted skin reaction caused by the active agent can be avoided.

In certain embodiments, it is preferred for the active agent-containing layer to be an active agent-containing matrix layer, preferably comprising
(i) an active agent, in particular in an amount of at least 0.5% by weight, based on the total amount of the active agent-containing layer,
(ii) a polymer I, in particular in an amount of 20 to 99% by weight, or from 60 to 99% by weight, based on the total amount of the active agent-containing layer, and optionally
(iii) a solubilizer, in particular in an amount of from 1 to 40% by weight, based on the total weight of the active agent-containing layer.

The active agent (and optionally the solubilizer) is preferably homogeneously distributed within the active agent-containing matrix layer. As used herein, an active agent-containing matrix layer is a layer containing the active agent dissolved or dispersed in the polymer I, or containing the active agent dissolved in the solubilizer to form an active agent-solubilizer mixture that is dispersed in the form of deposits (in particular droplets) in the polymer I. Thus, the active agent-containing matrix layer may comprise the active agent dissolved in solubilizer as microreservoir droplets distributed within the active agent-containing matrix layer. The proportion of the microreservoir droplets in the active agent-containing matrix layer is usually less than about 40% by weight, or less than about 35% by weight, or between about 20% and about 30% by weight.

In certain embodiments, the active agent-containing layer may comprise the active agent in an amount of at least 0.5% by weight, at least 1% by weight, at least 2% by weight, or at least 5% by weight, based on the total weight of the active agent-containing layer. In these or other embodiments, the active agent-containing layer may comprise the active agent in an amount of not more than 30% by weight, mot more than 20% by weight, not more than 15% by weight, or not more than 10% by weight, based on the total weight of the active agent-containing layer. In particular, the active agent-containing layer may comprise the active agent in an amount of from 0.5 to 30% by weight, from 1 to 20% by weight, from 2 to 15% by weight, or from 5 to 10% by weight.

In certain embodiments, the active agent-containing layer may comprise the polymer I in amount of from 20 to 99% by weight, or from 60 to 90% by weight, based on the total weight of the active agent-containing layer. It is to be understood that the aforementioned weight percent amounts refer to the overall amount of the polymer I. For example, if the polymer I is a mixture of polymers, the overall amount in the active agent-containing layer is from 20 to 99% by weight, based on the total weight of the active agent-containing layer.

### Active Agent

In accordance with the invention, the active agent-containing layer comprises an active agent.

The active agent may be any compound responsible for the therapeutic effect(s) of the medical patch comprising the active agent-containing layer structure comprising the active agent-containing layer. In particular, the active agent may be a topically active agent or a systemically active agent. In certain embodiments, the active agent may be at least one irritating, in particular skin irritating, active agent.

For example, the active agent may be an analgesic, such as a TRPV1 agonist.

According to particular embodiments, the active agent may be a capsaicin analog. As used herein, the term “capsaicin analog” refers to a chemical compound that provides a similar pharmacological effect as capsaicin, in particular in view of the TRPV1 receptor. Capsaicin ((6E)-N-[(4-hydroxy-3-methoxyphenyl)methyl]-8-methylnon-6-enamide) is an alkaloid found in the Capsicum family, and a potent agonist of the transient receptor potential cation channel subfamily V member 1 (TRPV1), better known as vanilloid receptor. This receptor is a nonselective cation channel that allows the transient influx of Ca²⁺ when activated during the detection and transduction of nociceptive stimuli. As calcium in the cell is one of the most versatile second messengers in numerous intracellular signaling pathways and TRPV1 permits cations to pass through the cell membrane and into the cell when activated, the resulting depolarization of the neurons stimulates it to signal the brain. A capsaicin analog is thus more precisely understood to mean a natural or synthetic molecule that is clearly demonstrated to be TRPV1 receptor agonist and/or that preserves all or a part of the aromatic catechol ring in its chemical structure.

In particular embodiments, the active agent is selected from the group consisting of further capsaicinoids found in the Capsicum family such as dihydrocapsaicin (N-[(4-hydroxy-3-methoxyphenyl)methyl]-8-methylnonanamide), nordihydrocapsaicin (N-[(4-hydroxy-3-methoxyphenyl)methyl]-7-methyloctanamide), homocapsaicin ((6E)-N-(4-hydroxy-3-methoxybenzyl)-8-methyldec-6-enamide), homodihydrocapsaicin (N-[(4-hydroxy-3-methoxyphenyl)methyl]-9-methyldecanamide), or pseudocapsaicin (N-[(4-hydroxy-3-methoxyphenyl)methyl]nonanamide, also referred to as nonivamide), natural capsaicin analogs, such as, e.g., capsiate ((E)-4-hydroxy-3-methoxybenzyl 8-methylnon-6-enoate), gingerol ((5S)-5-hydroxy-1-(4-hydroxy-3-methoxyphenyl) decan-3-one), piperine ((2E,4E)-5-(2H-1,3-benzodioxol-5-yl)-1-(piperidin-1-yl) penta-2,4-dien-1-one), or resiniferatoxin ([(1R,2R,6R,10S,11R,13R, 15R, 17R)-13-benzyl-6-hydroxy-4,17-dimethyl-5-oxo-15-(prop-1-en-2-yl)-12,14,18-trioxapentacyclo [11.4.1.0^{1,10}.0^{2,6}.0^{11,15}]octadeca-3,8-dien-8-yl]methyl 2-(4-hydroxy-3-methoxyphenyl)acetate), and synthetic capsaicin analogs, such as, e.g., arvanil ((5Z,8Z,11Z,14Z)—N-[(4-hydroxy-3-methoxyphenyl)methyl]-5,8,11,14-eicosatetraenamide), civamide ((N-[(4-hydroxy-3-methoxyphenyl)methyl]-8-methyl-(Z)-6-nonanamide), N-oleyl homovanillamide (N-(9Z-octadecenyl)-3-methoxy-4-hydroxyphenylacetamide), olvanil ((Z)—N-[(4-hydroxy-3-methoxyphenyl)methyl]octadec-9-enamide), palvanil (N-[(4-hydroxy-3-methoxyphenyl)methyl]hexadecanamide), and phenylacetylrinvanil ([(Z,7R)-18-[(4-hydroxy-3-methoxyphenyl)methylamino]-18-oxooctadec-9-en-7-yl] 2-phenylacetate).

According to specific embodiments, the active agent is resiniferatoxin. Resiniferatoxin (abbreviated RTX) is an ultrapotent irritant produced in the latex of Euphorbia plant species. It has a score of 16 billion Scoville heat units, making pure resiniferatoxin about 500 to 1000 times hotter than pure capsaicin. Thus, RTX is rather toxic and can inflict chemical burns in minute quantities. It causes severe burning pain in sub-microgram (less than 1/1,000,000th of a gram) quantities when ingested orally. However, as it is currently the most potent TRPV1 agonist known, showing approximately 500 times higher binding affinity for TRPV1 than capsaicin, RTX can ablate sensory neurons as a “molecular scalpel” to achieve permanent analgesia. The therapeutic window of RTX is broad, allowing for the full desensitization of pain perception and neurogenic inflammation without causing unacceptable side effects. Currently, intra-articular RTX is undergoing clinical trials to treat moderate-to-severe knee pain in patients with osteoarthritis. Similar targeted approaches may be useful in the management of post-operative pain or pain associated with severe burn injuries.

Thus, particular active agent-containing layers according to the invention comprise a capsaicin analog, such as resiniferatoxin. Accordingly, in particular embodiments, the medical patch according to the invention is for the (topical or transdermal) administration of a capsaicin analog, or for the (topical or transdermal) administration of resiniferatoxin.

In certain embodiments, the medical patch and in particular the active agent-containing layer does not comprise capsaicin.

The medical patch and in particular the active agent-containing layer comprises the active agent in a therapeutically effective amount. In certain embodiments, the active agent-containing layer may contain at least 0.10 mg/cm², at least 0.20 mg/cm², at least 0.50 mg/cm², or at least 1.0 mg/cm² of the active agent per area of release. In these or other embodiments, the active agent-containing layer may comprise less than 12.0 mg/cm², less than 10.0 mg/cm², less than 6.0 mg/cm², or less than 3.0 mg/cm² of the active agent per area of release. In particular, the active agent-containing layer may comprise from 0.10 to 12.0 mg/cm², from 0.20 to 10.0 mg/cm², from 0.50 to 6.0 mg/cm², or from 1.0 to 3 mg/cm² of the active agent per area of release.

### Polymer I

In accordance with the invention, the active agent-containing layer comprises a polymer I. This polymer may provide for sufficient cohesion and/or adhesion of the active agent-containing layer.

Polymers which are suitable as the polymer I in accordance with the invention are polymers allowing the active agent to concentrate to more than 0.1% by weight, in particular to a therapeutically effective amount, if necessary, by use of an appropriate solubilizer. Accordingly, in some embodiments, the saturation concentration of the active agent in the polymer I may be more than 1% by weight, more than 5% by weight, more than 10% by weight, or more than 20% by weight. In alternative embodiments, the saturation concentration of the active agent in the polymer I may be (effectively) less than 1% by weight, or less than 0.1% by weight, but may be increased to more than 1% by weight, more than 5% by weight, more than 10% by weight, or more than 20% by weight by the addition of one or more solubilizer(s).

The polymer I may be selected from pressure sensitive adhesive polymers. Thus, in certain embodiments, the polymer I may be a pressure sensitive adhesive or a mixture of pressure sensitive adhesives.

In certain embodiments, the polymer I may be a polymer or a mixture of polymers selected from the group consisting of acrylic polymers, silicone acrylic hybrid polymers, silicone-based polymers, and polymers based on natural or synthetic rubbers. In particular, the polymer I may be a polymer or a mixture of polymers selected from the group consisting of silicone-based polymers and polymers based on natural or synthetic rubbers.

In particular embodiments, the polymer I may be a polymer or a mixture of polymers selected from silicone-based polymers, in particular from polymers based on polysiloxanes. In more particular embodiments, the polymer I may be an amine-compatible polysiloxane.

In particular embodiments, the polymer I may be a polymer or a mixture of polymers selected from natural or synthetic rubbers, in particular from styrenic triblock copolymers and polyisobutylenes. Accordingly, the polymer I may be a styrenic triblock copolymer, such as a SIS block copolymer, or the polymer I may be a polyisobutylene. Further, the polymer may be a mixture of a styrenic triblock copolymer and polyisobutylene, in particular a mixture of a SIS block copolymer and polyisobutylene.

Suitable polymers I according to the invention are commercially available e.g. under the brand names BIO-PSA (pressure sensitive adhesives based on polysiloxanes) JSR-SIS (SIS block copolymer-based pressure-sensitive adhesives) and Oppanol™ (polyisobutylenes).

Additional polymers may also be added, e.g., to enhance cohesion of the active agent-containing layer.

According to some embodiments, the polymer I contained in the active agent-containing layer is different from the polymer II contained in the skin contact layer. According to other embodiments, the polymer I contained in the active agent-containing layer is the same as the polymer II contained in the skin contact layer. In such embodiments, as the saturation concentration of the active agent in the polymer I is negligible (as in the skin contact layer and thus in the polymer II), the active agent-containing layer further comprises a solubilizer.

### Solubilizer

The medical patch according to the invention, and in particular the active agent-containing layer advantageously may comprise a solubilizer.

As outlined above, a solubilizer is an agent that is able to increase the solubility of the active agent in the active agent-containing layer substantially, e.g. by at least 1 percentage point (with respect to the amount of active agent in % by weight in the active agent-containing layer) per 10% by weight, per 5% by weight, per 1% by weight, or per 0.5% by weight solubilizer added to the active agent-containing layer. This can be achieved by a substance or a mixture of substances having a relatively high solubility for the active agent. Thus, in certain embodiments, the solubilizer may be a substance or a mixture of substances in which the capsaicin or capsaicin analog has a solubility of at least 30% by weight, at least 40% by weight, or at least 45% by weight.

The solubilizer may also be released along with the active agent to further act as penetration enhancer. Without wishing to be bound by theory, it is believed that a solubilizer may also have a limited or reduced affinity to the polymer I contained in the active agent-containing layer, and that this way, the driving force of the active agent dissolved in the solubilizer out of the patch is kept high (but only appears when after application of the medical patch to the skin of the patient).

In certain embodiments, the solubilizer may be an amphiphilic solvent. Suitable amphiphilic solvents include butanediols, in particular 1,3-butanediol, dipropylene glycol, tetrahydrofurfuryl alcohol, diethylene glycol dimethyl ether, diethylene glycol monoethyl ether (DGME), diethylene glycol monobutyl ether, propylene glycol, carboxylic acid esters of tri-and diethylene glycol, polyethoxylated fatty alcohols of 6-18 C atoms or 2,2-dimethyl-4-hydroxymethyl-1,3-dioxolane, or any mixture thereof or mixtures of these solvents.

In particular embodiments, the solubilizer is dipropylene glycol, diethylene glycol monoethyl ether, or dimethyl isosorbide. Diethylene glycol monoethyl ether and dimethyl isosorbide are, e.g., available under the trade names Transcutol® and Dottisol®, respectively.

The solubilizer may also be selected from the group consisting of glycerol-, polyglycerol-, propylene glycol- and polyoxyethylene-esters of medium chain and/or long chain fatty acids, such as glyceryl monolinoleate, medium chain glycerides and medium chain triglycerides, non-ionic solubilizers made by reacting castor oil with ethylene oxide, and any mixtures thereof which may further contain fatty acids or fatty alcohols, cellulose and methylcellulose and derivatives thereof such as hydroxypropylcellulose and hypromellose acetate succinate, various cyclodextrins and derivatives thereof, non-ionic tri-block copolymers having a central hydrophobic chain of polyoxypropylene flanked by two hydrophilic chains of polyoxyethylene known as poloxamers, water-soluble derivatives of vitamin E, pharmaceutical graded or agglomerated spherical isomalt, a polyethylene glycol, polyvinyl acetate and polyvinylcaprolactame-based graft copolymer, also abbreviated as PVAc-PVCap-PEG and known as Soluplus®, purified grades of naturally derived castor oil, of polyethylene glycol 400, of polyoxyethylene sorbitan monooleate (such as polysorbate 80) or of glucono-delta-lactone, maize and potato starch, as well as any of soluble polyvinylpyrrolidones, but also insoluble/cross-linked polyvinylpyrrolidones such as crospovidones, mentioned herein. Further, also the permeation enhancers and/or crystallization inhibitors mentioned below can act as solubilizers.

In particular, suitable solubilizers may include mixtures of propylene glycol monoesters and diesters of fatty acids, which are commercially available e.g. under the brand name Capryol™, such as propylene glycol monocaprylate (type II), a mixture of propylene glycol esters of caprylic acid, mainly composed of monoesters and further comprising a small fraction of diesters with a ratio of not less than 90% monoesters and not more than 10% diesters (commercially available as Capryol™ 90 supplied by Gattefossé), and polyethylene glycol ethers, in particular polyethylene glycol fatty alcohol ethers such as those commercially available as Brij®, e.g. polyethylene glycol dodecyl ether with an average molecular weight Mn of about 362 commercially available as Brij® L4.

A certain minimum amount of solubilizer is advantageous in that it will help preventing recrystallization of the active agent in the active agent-containing layer. On the other hand, if the amount of solubilizer is too high, cohesion of the active agent-containing layer may be impaired, thus, a balance has to be found. In addition, with respect to the advantageous effect of preventing active agent recrystallization, a crystallization inhibitor and a solubilizer may complement each other. Finally, the amount of crystallization inhibitor and/or solubilizer needed for effective prevention of active agent re-crystallization also depends on the amount of the active agent present in the active agent-containing layer.

### Skin Contact Layer

As outlined in more detail above, the medical patch according to the present invention comprises an active agent-containing layer structure comprising inter alia a skin contact layer, wherein the skin contact layer is an adhesive layer which is directly attached to the active agent-containing layer.

The medical patch according to the present invention is in particular characterized by the saturation concentration of the active agent in the skin contact layer being less than 0.1% by weight, preferably as determined by the “sandwich method”. In certain embodiments, the saturation concentration of the active agent in the skin contact layer is less than 0.05% by weight, less than 0.02% by weight, or less than 0.01% by weight. Preferably, the saturation concentration of the active agent in the skin contact layer is about 0% by weight. The saturation concentration relates to the amount of active agent being present in the skin contact layer, based on the total weight of the skin contact layer.

In certain embodiments, the saturation concentration of the active agent in the skin contact layer is less than a concentration of the active agent resulting in any unintended adverse effect such as a skin irritation upon contact after a short period of time. Such a concentration may be determined empirically by in vivo testing, by observing whether an adverse effect such as any form of skin irritation (redness, erythema, itching or other skin reaction) occurs or does not occur after applying model adhesive layers with defined active agent concentrations on the skin for a short period of time, e.g., 5 seconds, 10 seconds, 30 seconds or 1 minute. In particular, different model layers representing a series of active agent concentrations may be tested to determine the highest acceptable saturation concentration which will not yet cause any unintended adverse effect such as a skin irritation reaction. On the other hand, whether or not a medical patch with a certain set of active agent and skin contact layer results in a saturation concentration not resulting in any adverse effect can be simply determined (without a series of different concentration) by testing a model adhesive layer which is saturated with the active agent, or said medical patch, i.e., by applying on the skin as outlined above.

In accordance with the invention, the skin contact layer shields the active agent contained in the active agent-containing layer from the skin of the patient or other applying/removing person before and/or after application of the medical patch. The skin contact layer thus needs to be substantially free of the active agent. This means that the skin contact layer is typically manufactured as a layer free of the active agent. However, due to the concentration gradient, the active agent usually may migrate from the active agent-containing layer to the skin contact layer over time, until an equilibrium is reached. This migration is, however, limited by the saturation concentration of the active agent in the skin contact layer. Accordingly, the skin contact layer does not allow the active agent to be present at a concentration of more than 0.1% by weight.

Thus, in certain embodiments, the skin contact layer comprises the active agent in an amount of less than 0.1% by weight, based on the total weight of the skin contact layer. In particular embodiments, the skin contact layer comprises the active agent in an amount of less than 0.01% by weight, based on the total weight of the skin contact layer.

In certain embodiments, the skin contact layer may comprise the polymer II in amount of at least 95% by weight, at least 99% by weight, or in an amount of about 100% by weight, based on the total weight of the skin contact layer. The amount of the polymer II in the skin contact layer may range from 50 to 95% by weight, 60 to 99% by weight or 75 to 100% by weight, based on the total weight of the skin contact layer. In particular, the skin contact layer may essentially consist of the polymer II. It is to be understood that the aforementioned weight percent amounts refer to the overall amount of the polymer II. For example, if the polymer II is a mixture of polymers, the overall amount in the skin contact layer is from 50 to 100% by weight, based on the total weight of the skin contact layer.

### Polymer II

In accordance with the invention, the skin contact layer comprises a polymer II. This polymer may be decisive for the adhesive properties of the skin contact layer and further may reduce skin irritation inter alia due to its resiliency

Polymers which are suitable as the polymer II in accordance with the invention are polymers allowing the active agent to concentrate to not more than 0.1% by weight, not more than 0.05% by weight, not more than 0.02% by weight, or not more than 0.01% by weight, i.e. polymers in which the active agent is substantially insoluble. Thus, according to certain embodiments, the polymer II may be a polymer or a mixture of polymers in which the active agent is substantially insoluble.

Accordingly, the solubility parameter of the polymer II may differ from, in particular may be lower than the solubility parameter of the active agent by at least 5.0 MPa^{1/2}, at least 6.0 MPa^{1/2}, at least 8.0 MPa^{1/2}, or at least 10.0 MPa^{1/2}. In particular, the solubility parameter of the polymer II may be less than 18.5 MPa^{1/2}, less than 18.0 MPa^{1/2}, less than 17.5 MPa^{1/2}, less than 17.0 MPa^{1/2}, less than 16.0 MPa^{1/2}, or less than 15.0 MPa^{1/2}, preferably as calculated by Small's method.

The polymer II may be selected from pressure sensitive adhesive polymers. Thus, in certain embodiments, the polymer II may be a pressure sensitive adhesive or a mixture of pressure sensitive adhesives.

In certain embodiments, the polymer II may be a polymer or a mixture of polymers selected from the group consisting of silicone acrylic hybrid polymers, silicone-based polymers, silicone gel adhesives, and polymers based on natural or synthetic rubbers. In particular, the polymer II may be a polymer or a mixture of polymers selected from the group consisting of silicone-based polymers and silicone gel adhesives.

In some embodiments, the polymer II may be a silicone gel adhesive.

In other embodiments, the polymer II may not be a silicone gel adhesive. Thus, according to such embodiments the skin contact layer does not comprise silicone gel adhesives.

Also, the polymer II may be polymer or a mixture of polymers selected from silicone-based polymers, in particular from polymers based on polysiloxanes such as an amine-compatible polysiloxanes, or the polymer II may be a polymer or a mixture of polymers selected from natural or synthetic rubbers, in particular from styrenic triblock copolymers and/or polyisobutylenes, such as an SIS block copolymer and/or polyisobutylene.

Suitable polymers II according to the invention are commercially available e.g. under the brand names Soft skin adhesives (two-part silicone adhesive that cures upon mixing the two components). Alternative, suitable polymer II according to the invention are commercially available e.g. under the brand names BIO-PSA (pressure sensitive adhesives based on polysiloxanes) JSR-SIS (SIS block copolymer-based pressure-sensitive adhesives) and Oppanol™ (polyisobutylenes).

Additional polymers may also be added, e.g., to enhance adhesion of the skin contact layer.

### Acrylic Polymer

The terms acrylic polymer and acrylate polymer are synonymously referred to polymers based on acrylates. In certain embodiments, the acrylic polymers are pressure-sensitive adhesives based on acrylates. Pressure-sensitive adhesives based on acrylates may also be referred to as acrylate-based pressure-sensitive adhesives, or acrylate pressure-sensitive adhesives.

Pressure-sensitive adhesives based on acrylates may be provided in the form of a solution with a solids content preferably between 30% and 60%. Acrylate-based pressure-sensitive adhesives may or may not comprise functional groups such as hydroxy groups, carboxylic acid groups, neutralized carboxylic acid groups and mixtures thereof. Corresponding commercial products are available e.g. from Henkel under the tradename Duro Tak®. Such acrylate-based pressure-sensitive adhesives are based on monomers selected from one or more of acrylic acid, 2-ethylhexylacrylate, glycidylmethacrylate, 2-hydroxyethylacrylate, methylacrylate, methylmethacrylate, butylacrylate, butylmethacrylate, t-octylacrylamide and vinylacetate, and are provided in ethyl acetate, heptane, n-heptane, hexane, methanol, ethanol, isopropanol, 2,4-pentanedione, toluene or xylene or mixtures thereof.

Specific acrylate-based pressure-sensitive adhesives are commercially available as:
Duro-Tak™ 387-2287 or Duro-Tak™ 87-2287 (a copolymer based on vinyl acetate, 2-ethylhexyl-acrylate, 2-hydroxyethyl-acrylate and glycidyl-methacrylate provided as a solution in ethyl acetate without cross-linking agent),
Duro-Tak™ 387-2516 or Duro-Tak™ 87-2516 (a copolymer based on vinyl acetate, 2-ethylhexyl-acrylate, 2-hydroxyethyl-acrylate and glycidyl-methacrylate provided as a solution in ethyl acetate, ethanol, n-heptane and methanol with a titanium cross-linking agent),
Duro-Tak™ 387-2051 or Duro-Tak™ 87-2051 (a copolymer based on acrylic acid, butylacrylate, 2-ethylhexylacrylate and vinyl acetate, provided as a solution in ethyl acetate and heptane),
Duro-Tak™ 387-2353 or Duro-Tak™ 87-2353 (a copolymer based on acrylic acid, 2-ethylhexylacrylate, glycidylmethacrylate and methylacrylate, provided as a solution in ethyl acetate and hexane),
   Duro-Tak™ 87-4098 (a copolymer based on 2-ethylhexyl-acrylate and vinyl acetate, provided as a solution in ethyl acetate).
Duro-Tak™ 387-9301 (a copolymer based on methyl acrylate, 2-ethylhexyl acrylate and t-octyl acrylamide, provided as a solution in ethyl acetate).

Accordingly, the acrylic polymer may be selected from acrylic polymers comprising functional groups wherein the functional groups are selected from hydroxyl groups, carboxylic acid groups, neutralized carboxylic acid groups and mixtures thereof. In certain embodiments, the functional groups are limited to hydroxyl groups. The acrylic polymer may not comprise carboxylic acid groups or neutralized carboxylic acid groups or both groups, or may not comprise acidic groups, or may comprise no functional groups.

Depending on the type of commercially available acrylic polymer used and depending on whether a cross-linking agent is added to the coating composition, the polymer in the finalized active agent-containing layer or skin contact layer is cross-linked (and preferably is cross-linked by an aluminium and/or a titanium cross-linking agent) or is not cross-linked by a cross-linking agent.

### Silicone Acrylic Hybrid Polymer

Silicone acrylic hybrid polymers comprise a polymerized hybrid species that includes silicone-based sub-species and acrylate-based sub-species that have been polymerized together. The silicone acrylic hybrid polymer thus comprises a silicone phase and an acrylic phase. In certain embodiments, the silicone acrylic hybrid polymer is a silicone acrylic hybrid pressure-sensitive adhesive.

Silicone acrylic hybrid pressure-sensitive adhesives are usually supplied and used in solvents like n-heptane and ethyl acetate. The solids content of the pressure-sensitive adhesives is usually between 30% and 80%. The skilled person is aware that the solids content may be modified by adding a suitable amount of solvent.

The weight ratio of silicone to acrylate in the silicone acrylic hybrid pressure-sensitive adhesive may be from 5:95 to 95:5, or from 20:80 to 80:20, or from 40:60 to 60:40, or the ratio of silicone to acrylate may be about 50:50.

Suitable silicone acrylic hybrid pressure-sensitive adhesives which are commercially available include the PSA series 7-6100 and 7-6300 manufactured and supplied in n-heptane or ethyl acetate by Dow Corning (7-610X and 7-630X; X=1 n-heptane-based/X=2 ethyl acetate-based). For example, the 7-6102 silicone acrylic hybrid PSA having a silicone/acrylate ratio of 50/50 is characterized by a solution viscosity at 25° C. and about 50% solids content in ethyl acetate of 2,500 cP and a complex viscosity at 0.1 rad/s at 30° C. of 1.0e7 Poise. The 7-6302 silicone acrylic hybrid PSA having a silicone/acrylate ratio of 50/50 has a solution viscosity at 25° C. and about 50% solids content in ethyl acetate of 1,500 cP and a complex viscosity at 0.1 rad/s at 30° C. of 4.0e6 Poise.

Depending on the solvent in which the silicone acrylic hybrid pressure-sensitive adhesive is supplied, the arrangement of the silicone phase and the acrylic phase providing a silicone or acrylic continuous external phase and a corresponding discontinuous internal phase is different. If the silicone acrylic hybrid pressure-sensitive adhesive is provided in n-heptane, the composition contains a continuous, silicone external phase and a discontinuous, acrylic internal phase. If the silicone acrylic hybrid pressure-sensitive adhesive is provided in ethyl acetate, the composition contains a continuous, acrylic external phase and a discontinuous, silicone internal phase. After evaporating the solvent in which the silicone acrylic hybrid pressure-sensitive adhesive is provided, the phase arrangement of the resulting pressure-sensitive adhesive film or layer corresponds to the phase arrangement of the solvent-containing adhesive coating composition. For example, in the absence of any substance that may induce an inversion of the phase arrangement in a silicone acrylic hybrid pressure sensitive adhesive composition, a pressure-sensitive adhesive layer prepared from a silicone acrylic hybrid pressure-sensitive adhesive in n-heptane provides a continuous, silicone external phase and a discontinuous, acrylic internal phase, a pressure-sensitive adhesive layer prepared from a silicone acrylic hybrid pressure-sensitive adhesive in ethyl acetate provides a continuous, acrylic external phase and a discontinuous, silicone internal phase. The phase arrangement of the compositions can, for example, be determined in peel force tests with pressure-sensitive adhesive films or layers prepared from the silicone acrylic hybrid PSA compositions which are attached to a siliconized release liner. The pressure-sensitive adhesive film contains a continuous, silicone external phase if the siliconized release liner cannot or can only hardly be removed from the pressure-sensitive adhesive film (laminated to a backing film) due to the blocking of the two silicone surfaces. Blocking results from the adherence of two silicone layers which comprise a similar surface energy. The silicone adhesive shows a good spreading on the siliconized liner and therefore can create a good adhesion to the liner. If the siliconized release liner can easily be removed the pressure-sensitive adhesive film contains a continuous, acrylic external phase. The acrylic adhesive has no good spreading due to the different surface energies and thus has a low or almost no adhesion to the siliconized liner.

The silicone acrylic hybrid polymer may be a silicone acrylic hybrid pressure-sensitive adhesive obtainable from a silicone-containing pressure-sensitive adhesive composition comprising acrylate or methacrylate functionality. It is to be understood that the silicone-containing pressure-sensitive adhesive composition comprising acrylate or methacrylate functionality can include only acrylate functionality, only methacrylate functionality, or both acrylate functionality and methacrylate functionality.

The silicone acrylic hybrid pressure-sensitive adhesive may comprise the reaction product of (a) a silicone-containing pressure-sensitive adhesive composition comprising acrylate or methacrylate functionality, (b) an ethylenically unsaturated monomer, and (c) an initiator. That is, the silicone acrylic hybrid pressure-sensitive adhesive is the product of the chemical reaction between these reactants ((a), (b), and (c)). In particular, the silicone acrylic hybrid pressure-sensitive adhesive may include the reaction product of (a) a silicone-containing pressure-sensitive adhesive composition comprising acrylate or methacrylate functionality, (b) a (meth)acrylate monomer, and (c) an initiator (i.e., in the presence of the initiator). That is, the silicone acrylic hybrid pressure-sensitive adhesive may include the product of the chemical reaction between these reactants ((a), (b), and (c)).

The reaction product of (a) a silicone-containing pressure-sensitive adhesive composition comprising acrylate or methacrylate functionality, (b) an ethylenically unsaturated monomer, and (c) an initiator may contain a continuous, silicone external phase and a discontinuous, acrylic internal phase or the reaction product of (a), (b), and (c) may contain a continuous, acrylic external phase and a discontinuous, silicone internal phase.

The silicone acrylic hybrid polymer may comprise a reaction product of a silicone polymer, a silicone resin and an acrylic polymer, wherein the acrylic polymer is covalently self-crosslinked and covalently bound to the silicone polymer and/or the silicone resin.

The silicone acrylic hybrid polymer may comprise a reaction product of a silicone polymer, a silicone resin and an acrylic polymer, wherein the silicone resin contains triorganosiloxy units R₃SiO_{1/2} where R is an organic group, and tetrafunctional siloxy units SiO_{4/2} in a mole ratio of from 0.1 to 0.9 R₃SiO_{1/2} units for each SiO_{4/2}.

The acrylic polymer may comprise at least an alkoxysilyl functional monomer, polysiloxane-containing monomer, halosilyl functional monomer or alkoxy halosilyl functional monomer. In certain embodiments, the acrylic polymer is prepared from alkoxysilyl functional monomers selected from the group consisting of trialkoxylsilyl (meth)acrylates, dialkoxyalkylsilyl (meth)acrylates, and mixtures thereof, or comprises end-capped alkoxysilyl functional groups. The alkoxysilyl functional groups may preferably be selected from the group consisting of trimethoxylsilyl groups, dimethoxymethylsilyl groups, triethoxylsilyl, diethoxymethylsilyl groups and mixtures thereof.

The acrylic polymer may also be prepared from a mixture comprising polysiloxane-containing monomers, preferably from a mixture comprising polydimethylsiloxane mono (meth)acrylate.

The silicone acrylic hybrid polymer may be prepared by a) reacting silicone polymer with silicone resin to form a resultant product, b) reacting the resultant product of a) with an acrylic polymer containing reactive functionality, wherein the components are reacted in an organic solvent.

The silicone acrylic hybrid polymer may be prepared by a) reacting a silicone resin with an acrylic polymer containing reactive functionality to form a resultant product, b) reacting the resultant product of a) with silicone polymer, wherein the components are reacted in an organic solvent.

The silicone acrylic hybrid polymer may be prepared by a) reacting a silicone polymer with an acrylic polymer containing reactive functionality to form a resultant product, b) reacting the resultant product of a) with silicone resin, wherein the components are reacted in an organic solvent.

Further suitable acrylic polymers, silicone resins, and silicone polymers that can be used for chemically reacting together a silicone polymer, a silicone resin and an acrylic polymer to provide a silicone acrylic hybrid polymer in accordance with the previous paragraphs are detailed in WO 2010/124187.

### Silicone-Based Polymer

The silicone-based polymer is a non-curing polymer, which is typically applied by a hot-melt or a solvent-based process and preferably does not undergo further curing to solidify.

Silicone-based polymers are based on polysiloxanes. They may therefore also be referred to as polymers based on polysiloxanes. Silicone-based polymers are generally obtainable by polycondensation of silanol endblocked polydimethylsiloxane with a silicate resin. Amine-compatible silicone-based polymers can be obtained by reacting the silicone-based polymer with trimethylsilyl (e.g. hexamethyldisilazane) in order to reduce the silanol content of the polymer and thus provide enhanced stability in the presence of amines. As a result, the residual silanol functionality is at least partly, preferably mostly or fully capped with trimethylsiloxy groups.

Thus, in particular embodiments, the silicone-based polymer may be an amine-compatible polysiloxane, obtainable by polycondensation of a silanol endblocked polydimethylsiloxane with a silicate resin followed by at least partial trimethylsilylation of the residual silanol functionality.

In certain embodiments, the silicone-based polymer may be a pressure sensitive adhesive based on polysiloxanes or a mixture of pressure sensitive adhesives based on polysiloxanes.

Pressure sensitive adhesives based on polysiloxanes provide for suitable tack and for quick bonding to various skin types, including wet skin, suitable adhesive and cohesive qualities, long lasting adhesion to the skin, a high degree of flexibility, a permeability to moisture, and compatibility to many actives and film-substrates. Such pressure sensitive adhesives are based on a resin-in-polymer concept wherein, by condensation reaction of silanol endblocked polydimethylsiloxane with a silica resin (also referred to as silicate resin), a pressure sensitive adhesive based on polysiloxane is prepared. For amine stability, the residual silanol functionality is additionally capped with trimethylsiloxy groups. The silanol endblocked polydimethylsiloxane content contributes to the viscous component of the visco-elastic behavior, and impacts the wetting and the spreadability properties of the adhesive. The resin acts as a tackifying and reinforcing agent, and participates in the elastic component. The correct balance between silanol endblocked polydimethylsiloxane and resin provides for the correct adhesive properties.

As indicated before, the tackiness of the silicone-based polymer may be modified by the resin-to-polymer ratio, i.e. the ratio of the silanol endblocked polydimethylsiloxane to the silicate resin, which is preferably in the range of from 50:50 to 70:30, or from 55:45 to 65:35. The tackiness will be increased with increasing amounts of the polydimethylsiloxane relative to the resin. High tack silicone-based polymers preferably have a resin-to-polymer ratio of 55:45, medium tack silicone-based polymers preferably have a resin-to-polymer ratio of 60:40, and low tack silicone-based polymers preferably have a resin-to-polymer ratio of 65:35.

According to certain embodiments, the silicone-based polymers may be obtainable by polycondensation of a silanol endblocked polydimethylsiloxane with a silicate resin, preferably with a resin-to-polymer ratio of from 50:50 to 70:30, or of 55:45, 60:40 or 65:35. Thus, in a particular embodiment, the silicone-based polymers may be a mixture of pressure sensitive adhesives obtainable by polycondensation of a silanol endblocked polydimethylsiloxane with a silicate resin with a resin-to-polymer ratio of 55:45 or of 60:40.

Further, according to certain embodiments, the polymers based on polysiloxanes may be a mixture of pressure sensitive adhesives with
a solution viscosity at 25° C. and about 60% solids content in heptane of 450 mPa s and/or a complex viscosity at 0.01 rad/s at 30° C. of 1×10⁸ Poise, and
a solution viscosity at 25° C. and about 60% solids content in heptane of 500 mPa s and/or a complex viscosity at 0.01 rad/s at 30° C. of 5×10⁶ Poise.

The silicone-based polymers are usually supplied and used in solvents like n-heptane, ethyl acetate or other volatile silicone fluids. The solids content of pressure sensitive adhesives based on polysiloxanes in solvents is usually between 60 and 85%, between 70 and 80%, or between 60 and 75%. The skilled person is aware that the solids content may be modified by adding a suitable amount of solvent.

High tack silicone-based polymers preferably have a complex viscosity at 0.01 rad/s and 30° C. of about 5×10⁶ Poise, medium tack silicone-based polymers preferably have a complex viscosity at 0.01 rad/s and 30° C. of about 5×10⁷ Poise, and low tack silicone-based polymers preferably have a complex viscosity at 0.01 rad/s and 30° C. of about 5×10⁸ Poise. High tack amine-compatible silicone-based polymers preferably have a complex viscosity at 0.01 rad/s and 30° C. of about 5×10⁶ Poise, medium tack amine-compatible silicone-based polymers preferably have a complex viscosity at 0.01 rad/s and 30° C. of about 5×10⁸ Poise, and low tack amine-compatible silicone-based polymers preferably have a complex viscosity at 0.01 rad/s and 30° C. of about 5×10⁹ Poise. Preferred pressure sensitive adhesives based on polysiloxanes in accordance with the invention are characterized by a solution viscosity at 25° C. and 60% solids content in n-heptane of more than about 150 mPa s, or from about 200 mPa s to about 700 mPa s, preferably as measured using a Brookfield RVT viscometer equipped with a spindle number 5 at 50 rpm. Theses may also be characterized by a complex viscosity at 0.01 rad/s at 30° C. of less than about 1×10⁹ Poise or from about 1×10⁵ to about 9×10⁸ Poise.

Suitable silicone-based polymers are commercially available under the brand names BIO-PSAs. Examples of silicone-based PSA compositions which are commercially available include the standard Liveo™ BIO-PSA series (7-4400,7-4500 and 7-4600 series) and the amine compatible (endcapped) Liveo™ BIO-PSA series (7-4100, 7-4200 and 7-4300 series) manufactured and typically supplied in n-heptane or ethyl acetate. For example, BIO-PSA 7-4201 is characterized by a solution viscosity at 25° C. and about 60% solids content in heptane of 450 mPa s and a complex viscosity at 0.01 rad/s at 30° C. of 1×10⁸ Poise. BIO-PSA 7-4301 has a solution viscosity at 25° C. and about 60% solids content in heptane of 500 mPas and a complex viscosity at 0.01 rad/s at 30° C. of 5×10⁶ Poise.

Pressure sensitive adhesives based on polysiloxanes may be obtained according to the following scheme:

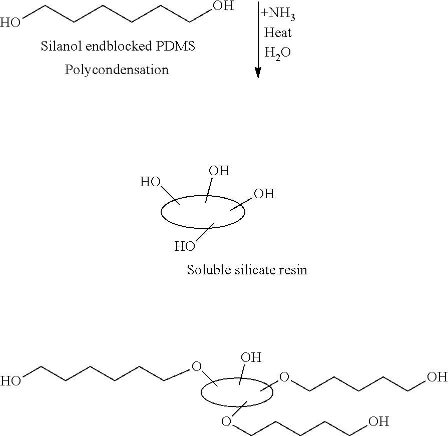

Such pressure sensitive adhesives based on polysiloxanes are available under the tradenames Liveo™ BIO-PSA 7-4401, BIO-PSA-7-4501, or BIO-PSA 7-4601, which are provided in the solvent n-heptane (indicated by the code “01”), or under the tradenames Liveo™ BIO-PSA 7-4402, BIO-PSA 7-4502, and BIO 7-4602, which are provided in the solvent ethyl acetate (indicated by the code “02”). Typical solids contents in the solvent are in the range of from 60 to 75%. The code “44” indicates a resin-to-polymer ratio of 65:35 resulting in a low tackiness, the code “45” indicates a resin-to-polymer ratio of 60:40 resulting in medium tackiness, the code “46” indicates a resin-to-polymer ratio of 55:45 resulting in high tackiness.

Amine-compatible pressure sensitive adhesives based on polysiloxanes may be obtained according to the following scheme:

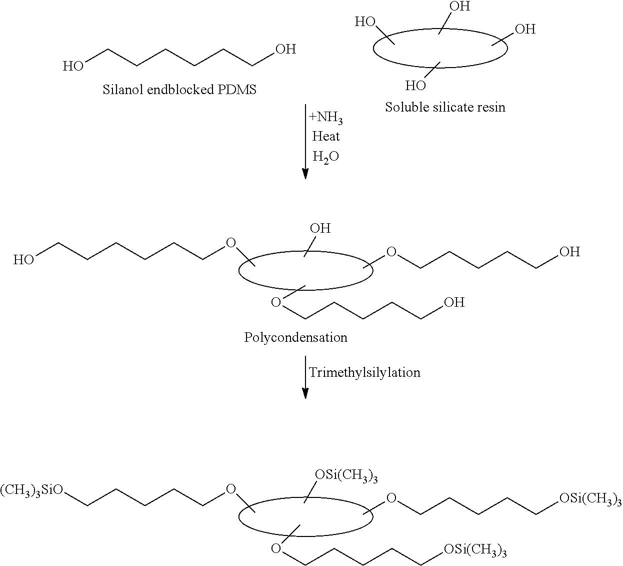

Such amine-compatible pressure sensitive adhesives based on polysiloxanes are available under the tradenames Liveo™ BIO-PSA 7-4101, BIO-PSA-7-4201, or BIO-PSA 7-4301, which are provided in the solvent n-heptane (indicated by the code “01”), or under the tradenames Liveo™ BIO-PSA 7-4102, BIO-PSA 7-4202, and BIO 7-4302, which are provided in the solvent ethyl acetate (indicated by the code “02”). Typical solids contents in the solvent are in the range of from 60 to 75%. The code “41” indicates a resin-to-polymer ratio of 65:35 resulting in a low tackiness, the code “42” indicates a resin-to-polymer ratio of 60:40 resulting in medium tackiness, the code “43” indicates a resin-to-polymer ratio of 55:45 resulting in high tackiness.

### Silicone Gel Adhesive

The silicone gel adhesive is an elastic, jelly-like material formed by lightly crosslinking silicone polymers. Thus, in contrast to the silicone-based polymers as used herein, the silicone gel adhesive is based on a curable gel producing composition. The silicone gel adhesive provides for the adhesiveness of the medical patch to the skin, while at the same time reducing the problem of skin irritation. Furthermore, the drug delivery of the medical patch is not negatively affected, surprisingly the skin permeation behavior is even improved.

Silicone gel adhesives are also referred to as silicone gels and, e.g., described in WO 2011/022199 A2.

The silicone gel adhesive is generally formed from linear or branched silicones having reactive groups thereon. Such reactive groups undergo a crosslinking reaction during curing. Examples of crosslinking reactions include the hydrosilylation reaction in which a silicone having an Si—H reactive group reacts with a silicone having an aliphatic unsaturated reactive group in the presence of a hydrosilylation catalyst. These materials are described, for example in U.S. Pat. Nos. 5,656,279, 5,891,076, EP 0 322 118 and U.S. Pat. No. 4,991,574 which are incorporated herein by reference. An alternative reaction is the condensation cure in which an alkoxy and/or hydroxy containing siloxanes are cured with a catalyst as described in U.S. Pat. No. 4,831,070 which is hereby incorporated by reference.

In certain embodiments, the silicone gel adhesives may be obtainable by reacting a gel producing composition comprising (i) at least one alkenyl-substituted polydiorganosiloxane, (ii) at least one organosiloxane, which contains silicone-bonded hydrogen atoms, and (iii) at least one catalyst for the reaction of the SiH groups with the Si-alkenyl groups. These compositions cure at normal ambient temperatures, but curing can be expedited by heating to elevated temperatures, e.g., from 40 to 140° C., or by applying UV light.

Suitable alkenyl groups contain from 2 carbon to about 6 carbon atoms and are exemplified by, but not limited to, vinyl, allyl, and hexenyl. The alkenyl groups in this component may be located at terminal, pendant (non-terminal), or both terminal and pendant positions. The remaining silicone-bonded organic groups in the alkenyl-substituted polydiorganosiloxane are independently selected from the group consisting of monovalent hydrocarbon and monovalent halogenated hydrocarbon groups free of aliphatic unsaturation. These groups typically contain from 1 carbon to about 20 carbon atoms, alternatively from 1 carbon to 8 carbon atoms and are exemplified by, but not limited to, alkyl such as methyl, ethyl, propyl, and butyl; aryl such as phenyl; and halogenated alkyl such as 3,3,3-trifluoropropyl. Typically, at least 50 percent of the organic groups in the alkenyl-substituted polydiorganosiloxane are methyl. The structure of the alkenyl-substituted polydiorganosiloxane is typically linear, however, it may contain some branching due to the presence of trifunctional siloxane units. The viscosity of the alkenyl-substituted polydiorganosiloxane can be any desired. For example, it can be >0 mm²/s to 100,000 mm²/s, alternatively 50 mm²/s to 80,000 mm²/s, alternatively 300 mm²/s-3,000 mm²/s.

Methods for preparing the alkenyl-substituted polydiorganosiloxanes (i) of the present invention, such as condensation of the corresponding halosilanes or equilibration of cyclic polydiorganosiloxanes, are well known in the art.

The alkenyl-substituted polydiorganosiloxanes can be used in the gel producing composition in an amount of 10 to 90% by weight, based on the weight of the composition, alternatively 40 to 90% by weight, alternatively 50 to 80% by weight. The amount of alkenyl groups present in the alkenyl-substituted polydiorganosiloxane is typically in the range of 0.05 to 1% by weight, alternatively 0.05 to 1% by weight, based on the weight of the alkenyl-substituted polydiorganosiloxane.

The organosiloxane containing silicone-bonded hydrogen atoms (ii) are also known in the art as described, for example in U.S. Pat. No. 3,983,298. The hydrogen atoms in this component may be located at terminal, pendant (non-terminal), or both terminal and pendant positions. The remaining silicone-bonded organic groups in this component are independently selected from the group consisting of monovalent hydrocarbon and monovalent halogenated hydrocarbon groups free of aliphatic unsaturation. These groups typically contain from 1 carbon to about 20 carbon atoms, alternatively from 1 carbon to 8 carbon atoms, and are exemplified by, but not limited to, alkyl such as methyl, ethyl, propyl, and butyl; aryl such as phenyl; and halogenated alkyl such as 3,3,3-trifluoropropyl. In one embodiment of the invention, at least 50 percent of the organic groups in the organosiloxane containing silicone-bonded hydrogen atoms are methyl. The structure of the organosiloxane containing silicone-bonded hydrogen atoms is typically linear however; it may contain some branching due to the presence of trifunctional siloxane units. The viscosity of the organosiloxane containing silicone-bonded hydrogen atoms can be any desired. For example, it can be >0 mm²/s to 100,000 mm²/s, alternatively, 5 mm²/s to 500 mm²/s.

Methods of preparing the organosiloxane containing silicone-bonded hydrogen atoms of the present invention by co-hydrolysis of the appropriate chlorosilanes are known in the art; U.S. Pat. No. 2,877,255 to Clark; Japanese Laid Open Patent Application (KOKAI) SHO 62 (1987)-39660 to Mogi et al.; and U.S. Pat. Nos. 5,446,185 and 5,493,040 to Cobb et al., which are all hereby incorporated by reference.

The organosiloxanes containing silicone-bonded hydrogen atoms can be used in the gel producing composition in an amount of 1 to 30% by weight, based on the weight of the composition, alternatively 5 to 20% by weight, and alternatively 5 to 15% by weight. In one embodiment, the amount of hydrogen group present in the organosiloxane containing silicone-bonded hydrogen atoms is between 0.05 to 1.44% by weight, based on the weight of the organosiloxane containing silicone-bonded hydrogen atoms.

In the gel producing compositions, (i) and (ii) are preferably present such that the ratio of (H as SiH):(Alkenyl as Si-Alkenyl) is generally in the range of 0.1:1 to 10:1.

The hydrosilylation catalyst (iii) promotes the addition reaction of the alkenyl-substituted polydiorganosiloxane with the organosiloxane containing silicone-bonded hydrogen. The hydrosilylation catalyst can be any of the well-known hydrosilylation catalysts comprising a platinum group metal, a compound containing a platinum group metal, or a microencapsulated platinum group metal or compound containing same. These platinum group metals include platinum, rhodium, ruthenium, palladium, osmium and iridium. Platinum and platinum compounds are preferred catalysts based on their high activity level in hydrosilylation reactions. One class of platinum catalysts is the complexes of chloroplatinic acid with certain vinyl-containing organosiloxane compounds disclosed by Willig in U.S. Pat. No. 3,419,593, which is hereby incorporated by reference. A specific catalyst of this type is the reaction product of chloroplatinic acid and 1,3-diethenyl-1,1,3,3-tetramethyldisiloxane.

The hydrosilylation catalyst is present in an amount sufficient to cure the composition of the present invention. Typically, the concentration of the catalyst is sufficient to provide from 0.1 ppm to 500 ppm (part per million), alternatively from 1 ppm to 100 ppm, alternatively from 1 ppm to 50 ppm of a platinum group metal, based on the weight of (i) and (ii).

In view of the above, in a particular embodiment, the silicone gel adhesives may be obtainable by reacting a gel producing composition comprising (i) a copolymer of vinylmethylsiloxane and dimethylsiloxane with (ii) methylhydrogen polysiloxane with trimethylsilyl endgroups in the presence of (iii) a platinum catalyst, wherein preferably (i) and (ii) are present such that the ratio of (H as SiH):(Alkenyl as Si-Alkenyl) is generally in the range of 0.1:1 to 10:1.

An optional ingredient is a hydroxy substituted silicone resin as described in US. Patent Application No. 2007-0202245, herein incorporated by reference. The resin is typically comprised of groups having the formula R³₃SiO_{1/2} (“M” groups) and groups having the formula SiO_{4/2} (“Q” groups) where R³ is a alkyl group having 1 carbon to 6 carbon atoms or alkylene group having 1 carbon to 6 carbon atoms, typically methyl or vinyl. If an alkenyl group is present in the resin, typically the mol-% of R groups present as alkenyl groups is <10 mol-%, alternatively 5 mol-%. The number ratio of M groups to Q groups is typically in the range of 0.6:1 to 4:1, alternatively 0.6:1 to 1.0:1. The silicone resin typically contains 0.1 to 5% by weight, alternatively 1.0 to 5% by weight silicone-bonded hydroxy groups.

The resin can be used in the gel producing composition in an amount of 2 to 45% by weight, based on the weight of the gel producing composition and resin; alternatively 5 to 40% by weight, alternatively 10 to 35% by weight.

Thus, in particular embodiments, the silicone gel adhesives may be a silicate resin-reinforced silicone gel adhesive that contains from about 2 to about 45% by weight of at least one hydroxyl substituted silicate resin.

In particular embodiments, the silicone gel adhesive is a 2-component silicone adhesive system that cures upon mixing the two components. An example of such two-part silicone adhesive which is commercially available includes the Liveo™ Soft Skin Adhesives (e.g. MG 7-9700, MG 7-9800, MG 7-9850 and MG 7-9900) provided as a kit including components A and B. It is a platinum-catalyzed, soft, fillerless elastomeric silicone adhesive for adhering medical devices to the skin with medium adhesion force and gentle removal. The two components A and B are preferably mixed in a ratio of 1:1.

The silicone gel adhesive layer can be made by processes known in the art. For example, the gel may be pre-formed (e.g. as a sheet) by molding, calendaring, extruding, spraying, brushing applying by hand, casting or coating on a substrate such as a liner. Or the silicone gel layer can be made by applying the gel producing composition to a substrate by spraying, coating, bar coating, etc. Once applied to the substrate the gel producing composition is cured to produce the silicone gel adhesive on the substrate.

### Polymer Based on Natural or Synthetic Rubbers

Polymers based on natural or synthetic rubbers include hydrocarbon polymers such as (natural and synthetic) polyisoprene, polybutylene and polyisobutylene, styrene/butadiene polymers, styrene-isoprene-styrene block copolymers, butyl rubber, halogen-containing polymers such as polyacrylic-nitrile, polytetrafluoroethylene, polyvinylchloride, polyvinylidene chloride, and polychlorodiene, other copolymers thereof. The polymers may in particular be used in combination with a tackifier as defined below.

According to certain embodiments, the polymer may be a styrenic triblock copolymer selected from the group consisting of styrene-ethylene-styrene (SES) block copolymers, styrene-butadiene-styrene (SBS) block copolymers, styrene-isoprene-styrene (SIS) block copolymers, styrene-ethylene/butylene-styrene (S-EB-S) block copolymers, styrene-ethylene/butylene/propylene-styrene (s-EBS-S) block copolymers, styrene-isoprene/butadiene-styrene (S-IB-S) block copolymers, and mixtures thereof.

In certain embodiments, the polymer may be at least one SIS block copolymer. The at least one SIS block copolymer may consist of three blocks of polystyrene, polyisoprene and polystyrene and in particular has a molecular weight of from about 100,000 to 200,000. In particular embodiments, the SIS block copolymer may comprise blocks of polystyrene and of polyisoprene in a ratio of from about 10:90(%) to about 30:70(%), or in a ratio of about 15:85(%) or about 22:78(%).

In other embodiments, the polymer is at least one polyisobutylene and may be a combination of two different types of polyisobutylenes, in particular a combination of low-molecular weight polyisobutylene and high-molecular weight polyisobutylene. In particular embodiments, the ratio of the low-molecular weight polyisobutylene to the high-molecular weight polyisobutylene is in the range of from 75:25 to 90:10.

Suitable SIS block copolymers according to the invention are commercially available e.g. under the brand names JSR-SIS. Specific SIS block copolymer-based pressure-sensitive adhesives are available under the tradenames JSR-SIS5229 and JSR-SIS5002.

Suitable polyisobutylenes according to the invention are commercially available e.g. under the tradename Oppanol®. Combinations of high-molecular weight polyisobutylenes (B100, B80) and low-molecular weight polyisobutylenes (B10, B11, B12, B13) may be used. Suitable ratios of low-molecular weight polyisobutylene to high-molecular weight polyisobutylene are in the range of from 100:1 to 1:100, from 95:5 to 40:60, or from 90:10 to 75:25. A particular example for a polyisobutylene combination is B10/B100 in a ratio of 85/15, or B12/B100 in a ratio of 80/20. Oppanol® B100 has a viscosity average molecular weight Mv of 1,110,000, and a weight average molecular weight Mw of 1,550,000, and an average molecular weight distribution Mw/Mn of 2.9. Oppanol® B10 has a viscosity average molecular weight Mv of 40,000, and a weight average molecular weight Mw of 53,000, and an average molecular weight distribution Mw/Mn of 3.2. Oppanol® B12 has a viscosity average molecular weight Mv of 55,000, and a weight average molecular weight Mw of 70,000, and an average molecular weight distribution Mw/Mn of 3.2. A suitable polyisobutylene adhesive is also commercially available e.g. under the brand name Duro-Tak™ 87-6908.

### Further Additives

The medical patch according to the invention, and in particular the active agent-containing and/or the skin contact layer may further comprise at least one additive or excipient. Said additives or excipients are preferably selected from the group consisting of crystallization inhibitors, fillers, substances for skincare, pH regulators, preservatives, tackifiers, softeners, stabilizers, and permeation enhancers, in particular from crystallization inhibitors, tackifiers, softeners, stabilizers, and permeation enhancers. Such additives may be present in the active agent-containing layer or skin contact layer in an amount of from 0.001 to 80% by weight, e.g. from 1 to 20% by weight or from 0.01 to 10% by weight, based on the total weight of the active agent-containing layer.

It should be noted that in pharmaceutical formulations, the formulation components are categorized according to their physicochemical and physiological properties, and in accordance with their function. This means in particular that a substance or a compound falling into one category is not excluded from falling into another category of formulation component. E.g. a certain polymer can be a crystallization inhibitor but also a tackifier. Some substances may e.g. be a typical softener but at the same time act as a permeation enhancer. The skilled person is able to determine based on his general knowledge in which category or categories of formulation component a certain substance or compound belongs to. In the following, details on the excipients and additives are provided which are, however, not to be understood as being exclusive. Other substances not explicitly listed in the present description may be as well used in accordance with the present invention, and substances and/or compounds explicitly listed for one category of formulation component are not excluded from being used as another formulation component in the sense of the present invention.

In certain embodiments, the medical patch, in particular the active agent-containing layer may further comprise a crystallization inhibitor. Suitable examples of crystallization inhibitors include polyvinylpyrrolidone, vinyl acetate/vinylpyrrolidone copolymer and cellulose derivatives. The crystallization inhibitor is preferably polyvinylpyrrolidone, more preferably soluble polyvinylpyrrolidone. The crystallization inhibitor may increase the solubility of the active agent or inhibit the crystallization of the active agent.

In certain embodiments, the medical patch, in particular the active agent-containing layer may further comprise a viscosity increasing agent. Adding such agents allows the active agent to be dispersed more easily, in particular in an appropriate solubilizer. The viscosity-increasing agent may be selected from the group consisting of cellulose derivatives and high molecular weight polyacrylic acids, and any mixture thereof, in particular ethyl cellulose.

In certain embodiments, the medical patch, in particular the active agent-containing layer may further comprise a stabilizer, wherein the stabilizer is preferably selected from tocopherol and ester derivatives thereof and ascorbic acid and ester derivatives thereof. Preferred stabilizers include sodium metabisulfite, ascorbyl esters of fatty acids such as ascorbyl palmitate, ascorbic acid, butylated hydroxytoluene, tocopherol, tocopheryl acetate and tocopheryl linoleate.

In certain embodiments, the medical patch, in particular the active agent-containing layer and/or the skin contact layer may further comprise a softener/plasticizer. Exemplary softeners/plasticizers include linear or branched, saturated or unsaturated alcohols having 6 to 20 carbon atoms, triglycerides and polyethylene glycols.

In certain embodiments, the medical patch, in particular the active agent-containing layer and/or the skin contact layer may further comprise a pH regulator. Suitable pH regulators include mild acids and bases including amine derivatives, inorganic alkali derivatives, and polymers with basic or acidic functionality.

In certain embodiments, the medical patch, in particular the active agent-containing layer and/or the skin contact layer may further comprise a preservative. Suitable preservatives include parabens, formaldehyde releasers, isothiazolinones, phenoxyethanol, and organic acids such as benzoic acid, sorbic acid, levulinic acid and anisic acid.

In certain embodiments, the medical patch, in particular the skin contact layer may further comprise a substance for skincare. Such substances may be used to avoid or reduce skin irritation as detectable by the dermal response score. Suitable substances for skincare include sterol compounds such as cholesterol, dexpanthenol, alpha-bisabolol, and antihistamines.

Fillers such as silica gels, titanium dioxide and zinc oxide may be used in conjunction with the medical patch, in particular the active agent-containing layer, in order to influence certain physical parameters, such as cohesion and bond strength, in the desired way.

In case the active agent-containing layer structure and in particular the skin contact layer is required to have self-adhesive properties and one or more polymers is/are selected which does/do not provide sufficient self-adhesive properties, a tackifier is added. The tackifier may be an alicyclic saturated hydrocarbon resin, a hydrogenated rosin glycerol ester, paraffinum liquidum, or a mixture thereof, in particular a mixture comprising an alicyclic saturated hydrocarbon resin and paraffinum liquidum, or a mixture comprising a hydrogenated rosin glycerol ester and paraffinum liquidum. Further, the tackifier may be selected from polyvinylpyrrolidone (which, due to its ability to absorb water, is able to maintain the adhesive properties of the matrix layer and thus can be regarded as a tackifier in a broad sense), triglycerides, polyethylene glycols, dipropylene glycol, resins, resin esters, terpenes, and derivatives thereof, ethylene vinyl acetate adhesives, dimethylpolysiloxanes and polybutenes, preferably polyvinylpyrrolidone and more preferably soluble polyvinylpyrrolidone.

In certain embodiments, the medical patch, in particular the active agent-containing layer, may further comprise a permeation enhancer. Permeation enhancers are substances, which influence the barrier properties of the stratum corneum in the sense of increasing the active agent permeability. Some examples of permeation enhancers are polyhydric alcohols such as dipropylene glycol, propylene glycol, and polyethylene glycol; oils such as olive oil, squalene, and lanolin; fatty ethers such as cetyl ether and oleyl ether, fatty acid esters such as isopropyl myristate; urea and urea derivatives such as allantoin, polar solvents such as dimethyldecylphosphoxide, methylcetylsulfoxide, dimethylaurylamine, dodecyl pyrrolidone, isosorbitol, dimethylacetonide, dimethylsulfoxide, decylmethylsulfoxide, and dimethylformamide, salicylic acid, amino acids, benzyl nicotinate, and higher molecular weight aliphatic surfactants such as lauryl sulfate salts. Other agents include oleic and linoleic acids, ascorbic acid, panthenol, butylated hydroxytoluene, tocopherol, tocopheryl acetate, tocopheryl linoleate, propyl oleate, and isopropyl palmitate. If the active agent-containing layer further comprises a permeation enhancer, the permeation enhancer is preferably selected from diethylene glycol monoethyl ether (Transcutol®), diisopropyl adipate, isopropyl myristate, isopropyl palmitate, lauryl lactate, and dimethylpropylene urea.

### Shape of Medical Patch

In some embodiments, the active agent-containing layer structure has a hexagonal or pentagonal shape which is provided by the backing layer, the active agent-containing layer and the additional skin contact layer respectively. The hexagonal shape comprises at least one hexagon, wherein in particular
all pairs of opposite sides of the hexagon are parallel, and
the sides of the hexagon have a length of from 0.2 to 10 cm.
   The pentagonal shape comprises at least one pentagon, wherein in particular
the sides of the pentagon have a length of from 0.2 to 12.5 cm.

The hexagonal shape may comprise one to ten, such as one, two, three, four, or five hexagons, wherein preferably the hexagons adjoin each other and/or do not overlap. The pentagonal shape may comprise one to ten, such as one, two, three, four, six or nine pentagons, wherein preferably the pentagons adjoin each other and/or do not overlap. The two or more hexagons or pentagons are preferably integrally connected to each other. Hexagonal or pentagonal shapes can but do not need to comprise perforations.

In certain embodiments, the at least one hexagon is at least one convex hexagon, and the hexagonal shape comprises at least one convex hexagon, wherein all pairs of opposite sides of the convex hexagon are parallel, and the sides of the convex hexagon have a length of from 0.2 to 10 cm. The hexagonal shape can comprise one or two convex hexagons, in particularly integrally connected to each other. In certain embodiments, the at least one pentagon is at least one convex pentagon, and the pentagonal shape comprises at least one convex pentagon, wherein the sides of the convex pentagon have a length of from 0.2 to 12.5 cm. The pentagonal shape can comprise one to three convex pentagons, in particular integrally connected to each other. In particular embodiments, the hexagonal shape is a convex hexagon, or the pentagonal shape is a convex pentagon. In some embodiments, the convex pentagon may be non-regular. In other embodiments, the convex pentagon may be regular.

The hexagonal or pentagonal shape of the active agent-containing layer structure is advantageous in terms of an easy and less time-consuming handling of a medical patch containing the active agent-containing layer structure. It allows for simplified coverage of skin areas without cutting before application, thus reducing the risk of contaminating the cutting tool or the fingers with active, and also the risk of contaminating the patch at the cut. Further, uneven or rounded skin surfaces may be covered without wrinkling, thus providing full adhesiveness, and even complicated areas, such as fingers or toes may be easily surrounded using the active agent-containing layer structure. In particular, where the hexagonal or pentagonal shape comprises at least one convex hexagon or pentagon, the hexagonal or pentagonal shape only requires a short side length in relation to the area provided, thus reducing the risk of detaching edges of the medical patch.

In one embodiment, the hexagonal or pentagonal shape is a double-hexagon or double-pentagonal formed of two identical convex hexagons or pentagons, respectively, sharing two adjacent vertices at their common side. The double-hexagon or double-pentagon may be obtainable by mirroring one convex hexagon or pentagon on one of its sides (mirror axis), wherein the mirror axis then includes the common side. The double-hexagon or double-pentagon may be dividable at the common side to obtain two equal convex hexagons or pentagons, which may be applied either together or separately. Thus, in a further embodiment, the hexagonal or pentagonal shape is a double-hexagon or double-pentagon formed of two identical convex hexagons or pentagons sharing two adjacent vertices at their common side, wherein said common side is perforated for easy tear-off. In another embodiment, the pentagonal shape is a triple-pentagon formed of three identical convex pentagons sharing two adjacent vertices and their common side in pairs. The triple-pentagon may be obtainable by mirroring the first convex pentagon on one of its sides (mirror axis 1), wherein mirror axis 1 then includes the common side shared by the first and second convex pentagon, and mirroring the second convex pentagon on one of its four other sides (mirror axis 2), wherein mirror axis 2 then includes the common side shared by the second and third convex pentagon. The triple-pentagon may be dividable at the common sides to obtain three equal convex pentagons, which may be applied either together or separately. Thus, in a particularly preferred embodiment, the pentagonal shape is a triple-pentagon formed of three identical convex pentagons sharing two adjacent vertices and their common side in pairs, wherein said common side is perforated for easy tear-off. The double- or tripe-pentagon as described above may be present, e.g., in the form of a hexagon, in particular in the form of a regular hexagon.

The hexagonal shapes, in particular the convex hexagons or the double-hexagons, may have mirror symmetry and/or rotational symmetry. The hexagonal shapes can be mirror symmetrical with at least one axis of symmetry, such as two, three or four axes of symmetry, in particular six axes of symmetry. Alternatively or additionally, hexagonal shapes can be rotational symmetrical having an order of at least 2, such as 3 or 4, in particular have 6-fold rotational symmetry. Thus, in certain embodiments, hexagonal shapes are mirror symmetrical with at least four axes of symmetry and/or have at least 4-fold rotational symmetry, in particular are mirror symmetrical with six axes of symmetry and additionally have 6-fold rotational symmetry. The pentagonal shapes, such as in the form of a regular hexagon, may have mirror symmetry and/or rotational symmetry. Preferred pentagonal shapes are mirror symmetrical with at least one axis of symmetry, such as two, three or four axes of symmetry, in particular six axes of symmetry. Alternatively or additionally preferred pentagonal shapes are rotational symmetrical having an order of at least 2, such as 3 or 4, in particular have 6-fold rotational symmetry. Thus, particularly preferred pentagonal shapes are mirror symmetrical with at least four axes of symmetry and/or have at least 4-fold rotational symmetry, in particular are mirror symmetrical with six axes of symmetry and additionally have 6-fold rotational symmetry.

Where the active agent-containing layer structure has a hexagonal shape, the hexagon, in particular convex hexagon, has three pairs of parallel opposite sites, which may be different or equal in length. The two sides of each pair of parallel opposite sides may be equal in length, i.e. the hexagon may be a parallelogon. The parallelogon may be obtained by elongating a parallelogram, having 2-fold rotational symmetry, or by elongating a rhombus, having 2-fold rotational symmetry and additionally being mirror symmetrical with two axes of symmetry.

In certain embodiments, the six sides of the hexagon, in particular convex hexagon, or the five sides of the pentagon, in particular convex pentagon, are equal in length, i.e. the hexagon or pentagon is equilateral.

Alternatively, the hexagon is non-equilateral and has three sides of equal length and three other sides of other equal length. The three sides of equal length and the three other sides of other equal length preferably alternate. Such hexagons are preferably mirror-symmetrical with three axes of symmetry. In another alternative, the hexagon is non-equilateral and has four sides of equal length and two other sides of other equal lengths. This includes in particular a hexagon obtained by elongating a rhombus.

In certain embodiments, the hexagon is non-equilateral and the ratio of shortest side to longest side is 1:4 or less, 1:3 or less, 1:2 or less, 1:1.5 or less, or is about 1:1.

The sides of the hexagon according to the invention have a length of from 0.2 to 10 cm. The sides of the hexagon can also have a length of from 0.3 to 8 cm, from 0.5 to 4 cm, from 0.8 to 3.5 cm, or from 0.9 to 2.0 cm. In particular, two, three, four or six sides of the hexagon can have a length of about 0.5 cm, about 0.9 cm, about 1.5 cm, about 1.8 cm, about 2.8 cm, or about 3.2 cm. For example,
two sides of the hexagon can have a length of about 0.5 cm, about 0.9 cm or about 1.5 cm, and four sides of the hexagon can have a length of about 1.8 cm, about 2.8 cm or about 3.2 cm, or
three sides of the hexagon can have a length of about 0.5 cm, about 0.9 cm or about 1.5 cm, and three sides of the hexagon can have a length of about 1.8 cm, about 2.8 cm or about 3.2 cm, or
four sides of the hexagon can have a length of about 0.5 cm, about 0.9 cm or about 1.5 cm, and two sides of the hexagon can have a length of about 1.8 cm, about 2.8 cm or about 3.2 cm.

The height of the hexagon may be in the range of from 0.3 to 17 cm, from 0.8 to 12.5 cm, from 1.3 to 6 cm, or from 1.5 to 3.5 cm. The width of the hexagon may be in the range of from 0.4 to 20 cm, from 1 to 15 cm, from 1.6 to 7 cm, or from 1.8 to 4 cm.

In certain embodiments, the hexagon has an aspect ratio (height-to-width-ratio) of 4:1 or less, 3:1 or less, 2:1 or less, 1.5:1 or less, or √3:2 or less. The aspect ratio of the hexagon can also be √3:2 or less.

Alternatively, the pentagon is non-equilateral and has four sides of equal length, or the pentagon is non-equilateral and has two sides of equal length and two other sides of other equal length. Such pentagons are preferably mirror symmetrical with one axis of symmetry, wherein the remaining side is divided by the axis of symmetry in the middle. The pentagon, in particular convex pentagon, according to the invention may have
five sides of equal length,
five sides of different length,
two sides of equal length and three side of other and different length,
three sides of equal length and two sides of other and different length,
three sides of equal length and two sides of other length,
four sides of equal length and one side of other length, or
one pair of sides of equal length and one other pair of sides of other equal length (the remaining side having a different length than the one pair of sides of equal length and the one other pair of sides of equal length).

In certain embodiments, the pentagon is non-equilateral and the ratio of a side (any side) to another side (any other side) is about 1:1, or about 1:2/√3, or about 1: √3, or about 1:2, or about 1:2√3. In some embodiments, the pentagon is non-equilateral and the ratio of shortest side to longest side is 1:2. In other embodiments, the pentagon is non-equilateral and the ratio of shortest side to longest side is 1:2√3.

The sides of the pentagon according to the invention have a length of from 0.2 to 12.5 cm. The sides of the pentagon may have a length of from 0.3 to 10 cm, from 0.6 to 5 cm, from 0.7 to 4.5 cm, or from 0.9 to 2.4 cm. In particular, one, two, three, four or five sides of the pentagon have a length of about 0.5 cm, about 1 cm, about 1.3 cm, about 1.7 cm, about 2 cm, about 2.5 cm, about 3.1 cm, or about 4.5 cm. For example,
two sides of the pentagon have a length of about 0.5 cm, about 1 cm or about 1.3 cm, two sides of the pentagon have a length of about 1.3 cm, about 1.7 cm, about 2 cm or about 2.5 cm, and the remaining side has a length of about 2.5 cm, about 3.1 cm or about 4.5 cm, or
two sides of the pentagon have a length of about 1 cm or about 1.3 cm, two sides of the pentagon have a length of about 1.3 cm, about 1.7 cm or about 2 cm, and the remaining side has a length of about 1.7 cm, about 2 cm, or about 2.4 cm.

The height of the pentagon may be in the range of from 0.4 to 16.5 cm, 0.9 to 8.5 cm, from 1.4 to 7.5 cm, or from 1.6 to 4.5 cm. The width of the pentagon may be in the range of from 0.4 to 15.5 cm, from 0.9 to 8 cm, from 1.3 to 7 cm, or from 1.5 to 4 cm.

The (convex) pentagon according to the invention may belong to Type 1-15 as described above. In particular, the (convex) pentagon may be a Type 1 pentagon, or a Type 3 pentagon, or a Type 4 pentagon.

In certain embodiments, two sides of the pentagon are parallel. In particular, the pentagon has two parallel sides of equal length and two other sides of the same or other equal length. Alternatively, none of the sides of the pentagon are parallel.

In certain embodiments, the pentagon is mirror symmetrical with at least one axis of symmetry. In particular embodiments, the pentagon is mirror symmetrical with at least one axis of symmetry and has two (parallel) sides of equal length as well as two other sides of the same or other equal length, wherein the remaining sides is divided by the axis of symmetry in the middle, and/or has two interior angles of 90° as well as three interior angles of 120°, wherein one of the latter is divided by the axis of symmetry in the middle. The pentagon may be a pentagon of type I or a pentagon of type II.

In certain embodiments, the hexagon, in particular convex hexagon, is equiangular. Such convex hexagons have each an interior angle equal to 120°. Alternatively, the hexagon is non-equiangular, and the smallest angle is 60° or larger, 80° or larger, 90° or larger, or 110° or larger. In particular, the smallest angle is 60° or larger, 80° or larger, 90° or larger, or 110° or larger, and less than 120°. Further, the hexagon may have two interior angles of equal size (smaller angles) and four other interior angles of other equal size (larger angles), wherein the smaller angles are about 90°.

In a yet further embodiment, the hexagon, in particular convex hexagon, is regular. Such regular hexagons are preferably mirror symmetrical with six axes of symmetry and additionally have a 6-fold rotational symmetry. In a certain embodiment, the hexagonal shape is a double-hexagon formed of two identical convex hexagons sharing two adjacent vertices at their common side, wherein the two identical convex hexagons are regular.

In certain embodiments, the pentagon, in particular convex pentagon, is non-equiangular, wherein preferably one interior angle and one other interior angle sum up to 180°. The one interior angle and the one other interior angle summing up to 180° may be adjacent or non-adjacent. In some embodiments, one interior angle is 60° and one other interior angle is 120°. Alternatively, one interior angle and one other interior angle are 90° each. Preferred (convex) pentagons according to the invention have two interior angles of 90° and at least one interior angle of 120°. Particularly preferred (convex) pentagons according to the invention have two interior angles of 90° and three interior angles of 120°.

In some very particular embodiments, the pentagon is a special pentagon of type I having the following properties:
The pentagon is mirror-symmetrical having exactly one axis of symmetry.
The pentagon has two adjacent interior angles of 90° and three adjacent interior angles of 120°.
The pentagon has two parallel sides of equal length and two other sides of other equal length as well as one remaining side, wherein
   the ratio of the two parallel sides of equal length to the two other sides of equal length is about 1:2, and/or
   the ratio of the two parallel sides of equal length to the remaining side is about 1:2√3, and/or
   the ratio of the two other sides of equal length to the remaining side is about 1: √3.

In other very particular embodiments, the pentagon is a special pentagon of type II having the following properties:
The pentagon is mirror-symmetrical having exactly one axis of symmetry.
The pentagon has two adjacent interior angles of 120° and two non-adjacent interior angles of 90° separated by another interior angle of 120°.
The pentagon has two sides of equal length and two other sides of other equal length as well as one remaining side, wherein
   the ratio of the two sides of equal length to the two other sides of equal length is about 1: √3, and/or
   the ratio of the two sides of equal length to the remaining side is about 1:2, and/or
   the ratio of the two other sides of equal length to the remaining side is about 1:2/√3.

### Sheet of Medical Patches

In certain embodiments of the present invention, and in particular where the active agent-containing layer structure has a hexagonal or pentagonal shape, the active agent-containing layer structure can also be used in a sheet of medical patches, which comprises a number of active agent-containing layer structures arranged on a release liner.

Such a sheet of medical patches comprises
two or more active agent-containing layer structures as described above and a release liner,
wherein the release liner is coextensive with the active agent-containing layer structures or extends beyond the boundary of the active agent-containing layer structure in all directions.

By using a number of active agent-containing layer structures, the surface of the skin area to be treated may be spanned by puzzling the active agent-containing layer structures side by side without wrinkling. In doing so, where the active agent-containing layer structure has a hexagonal or pentagonal shape, such a shape allows for avoiding gaps and/or overlaps.

The number of active agent-containing layer structures provided in a sheet of medical patches to be peeled-off from the release liner depends on the size(s) of the active agent-containing layer structures. Suitable sheets of medical patches may comprise, e.g., from 2 to 400, from 3 to 300 or from 4 to 300, from 4 to 120 or from 6 to 120, or 6 to 30 or 8 to 30 active agent-containing layer structures. In certain embodiments, the sheet of medical patches comprises 2 to 15 or 150 to 300 active agent-containing layer structures. In further embodiments, the sheet of medical patches comprises 2, 3, 4, 5, 6, 7 or 8 active agent-containing layer structures. Alternatively, the sheet of medical patches may comprise 150, 180, 200, 240 or 300 active agent-containing layer structures. The active agent-containing layer structures may be equal or different.

The active agent-containing layer structures may be arranged on the release liner in any pattern, either adjoining each other (tiling the plane) or providing tiny gaps to facilitate grabbing of single active agent-containing layer structures. In some embodiments, the active agent-containing layer structures are arranged on the release liner in a space-saving manner. In particular, the active agent-containing layer structures are arranged side by side on the release liner. Thus, the active agent-containing layer structurers may be arranged in two or more parallel rows with respect to the longitudinal axis of the release liner, wherein each row may comprise from 2 to 20, from 3 to 12, or from 4 to 8 active agent-containing layer structures. For example, the active agent-containing layer structures may be arranged in 20 rows, wherein each row comprises 15 active agent-containing layer structures, in particular 15 equal active agent-containing layer structures. According to another example, the active agent-containing layer structures may be arranged in 4 rows, wherein each row comprises 3 to 6 active agent-containing layer structures, in particular 3 to 6 equal active agent-containing layer structures. In other embodiments, the active agent-containing layer structures are arranged in one or more types of recurring geometric pattern, wherein preferably each geometric pattern comprises from 2 to 15, from 3 to 12, or from 4 to 9 active agent-containing layer structures. Such types of geometric pattern may comprise polygons, such as hexagons. Thus, the active agent-containing layer structures may be arranged in the form of one or more hexagons, in particular regular hexagons, wherein preferably each of the (regular) hexagons comprises 2, 3, 4 or 9, in particular 2 or 3 active agent-containing layer structures. In particular embodiments, the active agent-containing layer structures may be arranged in the form of one or more regular hexagons, wherein each of the regular hexagons comprises two special pentagons of type I as described above, or each of the regular hexagons comprises three special pentagons of type II as described above.

In a specific embodiment, the active agent-containing layer structures tile the plane, in particular monohedrally tile the plane, such as in a prismatic pentagonal tiling or a Cairo pentagonal tiling. Active agent-containing layer structures tiling the plane can be parallelogons, in particular regular hexagons. The active agent-containing layer structures may also have hexagonal shapes selected from regular hexagons and/or double-hexagons formed of two identical regular hexagons sharing two adjacent vertices and their common side. In addition, active agent-containing layer structures tiling the plane are Type 1-15 pentagons, in particular Type 1 pentagons, or Type 3 pentagons, or Type 4 pentagons, such the (special) pentagons of type I or type II as described above. The active agent-containing layer structures may also have pentagonal shapes selected from double-pentagons formed of two identical convex pentagons sharing two adjacent vertices and their common side and/or triple-pentagons formed of three identical convex pentagons sharing two adjacent vertices and their common side in pairs, which may be present in the form of (regular) hexagon. The active agent-containing layer structures may be separated from each other or connected to each other. They may adjoin each other by sharing a common vertex in pairs and/or in threes and/or in fours. If the common vertex is shared in pairs only, this shared vertex may lie on a side, in particular centrally on a side, of a third pentagon.

In certain embodiments, the active agent-containing layer structures adjoin each other by sharing two adjacent vertices and their common side and are separated from each other by the common side being cut for independent peel-off from the release liner. Alternatively, the active agent-containing layer structures can adjoin each other by sharing two adjacent vertices and their common side and are connected to each other by the common side, which is weakened for easy tear-off. In particular, the common side can be perforated for easy tear-off. The sheet of medical patches may also comprise the active agent-containing layer structures adjoining each other by sharing two adjacent vertices and their common sides, wherein some of which are separated from each other by the common side being cut for independent peel-off from the release liner, and some of which are connected to each other by the common side which is weakened for easy tear-off. For example, the active agent-containing layer structures may be arranged in two or more parallel rows with respect to the longitudinal axis of the release liner, wherein each row comprises from 2 to 20 active agent-containing layer structures that adjoin each other by sharing two adjacent vertices and their common side, wherein the rows are separated from each other for independent peel-off and the active agent-containing layer structures within a row are connected to each other by the common side, which is perforated for easy tear-off. In certain embodiments, all active agent-containing layer structures are separated from each other by the common side being cut for independent peel-off from the release liner.

In certain embodiments, the active agent-containing layer structures adjoin each other by sharing at least one vertex and at least a part of an adjacent side and are separated from each other by the adjacent side being cut for independent peel-off from the release liner. Alternatively, the active agent-containing layer structures adjoin each other by sharing at least one vertex and at least a part of an adjacent side and are connected to each other by the adjacent side, which is weakened for easy tear-off. In particular, the adjacent side is perforated for easy tear-off. In some embodiments, the active agent-containing layer structures adjoin each other by sharing two adjacent vertices and their common side and are separated from each other by the common side being cut for independent peel-off from the release liner. Alternatively, the active agent-containing layer structures adjoin each other by sharing two adjacent vertices and their common side and are connected to each other by the common side, which is weakened for easy tear-off. In particular, the common side is perforated for easy tear-off. The sheet of medical patches may also comprise the active agent-containing layer structures adjoining each other by sharing at least one vertex and at least a part of an adjacent side or two adjacent vertices and their common sides, wherein some of which are separated from each other by the adjacent side or the common side being cut for independent peel-off from the release liner, and some of which are connected to each other by the adjacent side or the common side which is weakened for easy tear-off. In certain embodiments, all active agent-containing layer structures are separated from each other by the adjacent side or the common side being cut for independent peel-off from the release liner. For example, the active agent-containing layer structures may be arranged in two or more parallel rows with respect to the longitudinal axis of the release liner, wherein each row comprises from 2 to 20 active agent-containing layer structures that adjoin each other by sharing at least one vertex and at least a part of an adjacent side and/or two adjacent vertices and their common side, wherein the rows are separated from each other for independent peel-off and the active agent-containing layer structures within a row are connected to each other by the common side, which is perforated for easy tear-off. In another example, the active agent-containing layer structures may be arranged in the form of two or more regular hexagons, wherein each hexagon comprises 2 or 3 or 9 active agent-containing layer structures that adjoin each other by sharing at least one vertex and at least a part of an adjacent side and/or two adjacent vertices and their common side, wherein the hexagons are separated from each other for independent peel-off and the active agent-containing layer structures within a hexagon are connected to each other by the common side, which is perforated for easy tear-off.

In certain embodiments, the active agent-containing layer structures are connected to each other by at least one and preferably two or more common fastening bridges for joint peel-off from the release liner. The fastening bridge(s) may provide(s) (a) single point(s), at which the active agent-containing layer structures are connected to each other, even if they are separated from each other by the adjacent side or the common side being cut at least in part. This enables joint peel-off of the thus connected active agent-containing layer structures from the release liner to be applied to the skin of the patient, which is particularly advantageous in case of a large number of and/or small area active agent-containing layer structures. Alternatively, some fastening bridge(s) may be undone, e.g. teared off, for joint peel-off of a lower number of thus connected active agent-containing layer structures.

In further embodiments, the common fastening bride is provided at a vertex and connects at least two or at least three active agent-containing layer structures. Alternatively, the common fastening bridge may be provided at a side and connect two active agent-containing layer structures. In a certain sheet of medical patches, neighboring active agent-containing layer structures are all connected to each other in pairs by at least two common fastening bridges provided at two adjacent vertices or at their common side, such as at the two adjacent vertices. In particular, neighboring active agent-containing layer structures are all connected to each other in threes by a common fastening bridge provided at the common vertex. This relates in particular to active agent-containing layer structures tiling the plane. Thus, in one embodiment, the active agent-containing layer structures are regular hexagons and are connected to each other by at least one fastening bridge for joint peel-off from the release liner, wherein neighboring active agent-containing layer structures are all connected to each other in threes by a common fastening bridge provided at the common vertex.

In yet further embodiments, the common fastening bride is provided at a vertex and/or a side and connects at least two and preferably three or four active agent-containing layer structures, such as
two active agent-containing layer structures connected by two common fastening bridge provided at two adjacent vertices, or
three active agent-containing layer structures connected by one common fastening bridge provided at a shared vertex of two active agent-containing layer structures lying on a side of the third active agent-containing layer structure, or at the common vertex, or
four self-adhesive layer structures connected by one common fastening bride provided at the common vertex.

In a particular sheet of medical patches, neighboring active agent-containing layer structures are all connected to each other in pairs by at least two common fastening bridges provided at two adjacent vertices or at their common side and/or provided at one vertex and an adjacent side, preferably at the two adjacent vertices. In particular, neighboring active agent-containing layer structures are all connected to each other in threes or fours by a common fastening bridge provided at a vertex and/or a side, in particular at the common vertex. This relates in particular to active agent-containing layer structures tiling the plane. Thus, in a preferred embodiment of the sheet of medical patches according to the invention, the active agent-containing layer structures are (special) pentagons of type I or type II as described above and are connected to each other by at least one fastening bridge for joint peel-off from the release liner, wherein neighboring active agent-containing layer structures are all connected to each other in threes or fours by a common fastening bridge provided at a shared vertex lying on a side or at the common vertex.

In certain embodiments, the active agent-containing layer structures have hexagonal and/or pentagonal shapes selected from two or three different shapes in total. In further embodiments, the active agent-containing layer structures have hexagonal shapes comprising convex hexagons and double-hexagons formed of two identical convex hexagons sharing two adjacent vertices and their common side, in particular regular hexagons and double-hexagons formed of two identical regular hexagons, or the active agent-containing layer structures have pentagonal shapes comprising convex pentagons and double-pentagons formed of two identical convex pentagons sharing two adjacent vertices and their common side, in particular (special) pentagons of type I as described above and double-pentagons formed of two identical (special) pentagons of type I as described above. Alternatively, the active agent-containing layer structures all have the same hexagonal or pentagonal shape. In particular embodiments, the active agent-containing layer structures are double-hexagons formed of two identical convex hexagons sharing two adjacent vertices and their common side, in particular double-hexagons formed of two identical regular hexagons. In this context, the (regular) convex hexagons or the double-hexagons formed of two identical (regular) convex hexagons, respectively, are congruent. In particular embodiments, the active agent-containing layer structures are non-equilateral convex pentagons having two interior angles of 90° and three interior angles of 120°, and two parallel sides of equal length and two other sides of other equal length, wherein the ratio of the shortest side to the longest side is about 1:2√3. Alternatively, the active agent-containing layer structures are non-equilateral convex pentagons having two interior angles of 90° and three interior angles of 120°, and two sides of equal length and two other sides of other equal length, wherein the ratio of the shortest side to the longest side is about 1:2. In particular, the active agent-containing layer structures are (special) pentagons of type I or type II as described above, which may be arranged in the form of regular hexagons.

In certain embodiments, the sheet of medical patches (all medical patches of the sheet as a whole) has an area of release of from 1 cm² to 300 cm².

### Method of Treatment/Medical Use

The medical patch according to the present invention is suitable for use in a method of treatment, and in particular in a method of treating a human patient.

In certain embodiments, the medical patch according to the invention is for use in a method of treatment, wherein the medical patch is preferably applied to the skin of the patient for less than 90 minutes, less than 60 minutes, or less than 30 minutes. In certain embodiments, the medical patch according to the invention is for use in a method of treatment with a dosing interval of at least or about 1.5 months, at least or about 2 months, or at least or about 3 months. Thus, it is preferred for a medical patch to be only applied after a break of at least 90 days after removal a preceding medical patch.

In certain embodiment, the medical patch according to the invention is for use in a method of treating pain. In particular, the medical patch according to the invention is for use in a method of treating neuropathic pain, such as chronic neuropathic pain, preferably including postherpetic neuralgia, post-surgical neuralgia such as, e.g., post-herniotomy pain, post-thoracotomy pain or post-mastectomy pain, post-traumatic neuropathy, polyneuropathy such as, e.g., painful diabetic neuropathy, chemotherapy-induced neuropathy, neuropathy caused by tumors, HIV-associated neuropathy, alcohol-related neuropathy, small-fiber neuropathy or complex regional pain syndrome, radiculopathy, or compression syndromes such as carpal tunnel syndrome, or in a method of treating peripheral neuropathic pain, neuropathic pain associated with postherpetic neuralgia or diabetic peripheral neuropathy (DPN) of the hands and feet, or post-surgical neuropathic pain, and/or the medical patch according to the invention is for use in a method of treating nociceptive pain, such as acute nociceptive pain, preferably including somatic pain, or visceral pain.

In particular embodiments, the medical patch according to the invention is for use in a method of treating joint pain, or cancer pain. In particular, the medical patch according to the invention is for use in a method of treating pain associated with an articular condition, in particular associated with arthritis, such as, e.g., infectious or non-infectious arthritis, preferably in a method of treating pain associated with rheumatoid arthritis, juvenile arthritis, psoriatic arthritis, or osteoarthritis, such as e.g. hip arthritis, knee arthritis, feet arthritis, or arthritis of the finger joint.

In connection with the above, the medical patch according to the invention is preferably applied to at least one body surface on the patient, in particular selected from the back, the bottom, the hip, the legs, the knees, the feet, or the hands. The preferred application time of a medical patch according to the invention is less than or about 60 minutes on the back, bottom, hip or legs, and less than or about 30 minutes on the knees, feet or hands.

In particular embodiments, the medical patch according to the invention is not applied/removed by medical professionals, in particular the medical patch according to the invention is applied/removed by the patient itself.

According to a specific aspect, the present invention is also related to the use of the medical patch as described herein for the manufacture of a medicament, in particular for the manufacture of a medicament for treating pain, such as for the manufacture of a medicament for treating neuropathic pain, or nociceptive pain, or for the manufacture of a medicament for treating joint pain, or cancer pain. According to a more specific aspect, the present invention is also related to the use of the medical patch as described herein for the manufacture of a medicament for treating pain associated with an articular condition, such as arthritis.

According to a specific aspect, the present invention is also related to a method of treatment, in particular a method of treating pain, such as a method of treating neuropathic pain, or nociceptive pain, or a method of treating joint pain, or cancer pain, including applying a medical patch as described herein to the skin of a patient. According to a more specific aspect, the present invention is also related to a method of treating pain associated with an articular condition, such as arthritis, including applying a medical patch as described herein to the skin of a patient.

### Process of Manufacture

The medical patch according to the present invention may be manufactured using a process comprising the steps of
i. preparing the active agent-containing layer;
ii. preparing the skin contact layer; and
iii. laminating the skin contact layer onto the active agent-containing layer to obtain the active agent-containing layer structure.

The preparation of the active agent-containing layer may be performed before or after the preparation of the skin contact layer, or the preparation of the two layers may be performed in parallel before lamination. Alternatively, the active agent-containing layer is prepared first and the skin contact layer is directly prepared onto the active agent-containing layer, or the skin contact layer is prepared first and the active agent-containing layer is directly prepared onto the skin contact layer.

In some embodiments, the invention relates to a method of manufacturing an active agent-containing layer structure of a medical patch as described herein, comprising the steps of:
1) combining at least the components
   1. active agent, and
   2. polymer I,
   to obtain an active agent-containing coating composition;
2.1) coating the active agent-containing coating composition on a first intermediate liner; and
2.2) drying the coated active agent-containing coating composition to form the active agent-containing layer; and optionally
2.3) laminating the active agent-containing layer obtained by drying the active agent-containing coating composition coated on the first intermediate liner with the backing layer.

The method may further comprise the steps of:
3.1) coating an active agent-free coating composition comprising at least the polymer II on a second intermediate liner; and
3.2) drying or curing the coated active agent-free coating composition to form the skin contact layer; and optionally
3.3) laminating the skin contact layer obtained by drying or curing the active agent-free coating composition coated on the second intermediate liner with a release liner.

The method may further comprise the steps of:
4.1) removing the first and second intermediate liner from the active agent-containing layer and the skin contact layer; and
4.2) laminating the open side of the skin contact layer onto the open side of the active agent-containing layer to obtain the active agent-containing layer structure.

In other embodiments, the invention relates to a method of manufacturing an active agent-containing layer structure of a medical patch as described herein, comprising the steps of:
1) combining at least the components
   1. active agent, and
   2. polymer I,
   to obtain an active agent-containing coating composition;
2.1) coating the active agent-containing coating composition on the backing layer; and
2.2) drying the coated active agent-containing coating composition to form the active agent-containing layer; and optionally
3.1) coating an active agent-free coating composition comprising at least the polymer II on a release liner; and
3.2) drying or curing the coated active agent-free coating composition to form the skin contact layer; and optionally
4) laminating the open side of the skin contact layer onto the open side of the active agent-containing layer to obtain the active agent-containing layer structure.

The active agent-containing layer and the skin contact layer are preferably prepared separately as indicated above, and then laminated together by removing the intermediate liners (if any) and then laminating the open sides of the two layers together, so as to give an active agent-containing layer structure. Alternatively, the active-free coating composition is directedly coated and dried or cured onto the active agent-containing layer or the active agent-containing coating composition is directedly coated and dried onto the skin contact layer, prior to applying a release liner or the backing layer, respectively.

In this process of manufacture, in step 1) the components are preferably combined in a solvent to obtain the coating composition. The solvent may be selected from alcoholic solvents, in particular methanol, ethanol, isopropanol and mixtures thereof, and from non-alcoholic solvents, in particular ethyl acetate, n-propyl acetate, hexane, n-heptane, petroleum ether, toluene, and mixtures thereof, preferably from non-alcoholic solvents such as ethyl acetate or n-heptane.

The active agent may be homogeneously dissolved or dispersed in the active agent-containing coating composition. It may be provided in an amphiphilic solvent, which must not mix or may only mix to a small extend with the solvent of the active agent-containing coating composition.

The polymer I and/or the polymer II may be non-curing and therefore typically applied by a solvent-based process. Accordingly, the polymer I and/or the polymer II may be provided in a solvent, wherein the solids content in the solvent is preferably from 40 to 75% by weight. The solvent may be selected from alcoholic solvents and non-alcoholic solvents as described above.

The coated active agent-containing coating composition and/or active agent-free coating composition may be solidified by drying 2.2) or 3.2) to form the active agent-containing layer or skin contact layer, respectively. Drying is preferably performed at a temperature of from 20 to 90° C. Alternatively, the skin contact layer may be formed upon curing, i.e. crosslinking which is preferably performed at a temperature of from 40° C. to 140° C.

### EXAMPLES

The present invention will now be more fully described with reference to the accompanying examples. It should be understood, however, that the following description is illustrative only and should not be taken in any way as a restriction of the invention. Numerical values provided in the examples regarding the amount of ingredients in the composition or the area weight may vary slightly due to manufacturing variability.

### Example 1

The skin permeation rate and utilization of capsaicin for two medical patches comprising capsaicin were determined by in vitro experiments.

The two patches differ from each other only by the presence or absence of an additional skin contact layer. They were thus prepared equally, except that the steps of preparing and coating an active-free coating composition and laminating the resulting active-free layer with the previously prepared capsaicin-containing layer have been carried out only for one of the two patches, but not for the other.

### Capsaicin-Containing Coating Composition

For both patches, the formulation of the capsaicin-containing coating composition is summarized in Table 1.1 below. The solids %-values refer to the amounts (Amt) in % by weight.

TABLE 1.1

| Ingredient (Trade Name) | Solids [%] |
|---|---|
| Capsaicin | 8 |
| Polysiloxanes in n-heptane. Solids content of | 70 |
| 70% by weight (Liveo ™ BIO-PSA 7-4301 | |
| and BIO-PSA 7-4201) | |
| Transcutol (diethylene glycol monoethyl ether) | 19.2 |
| Ethyl cellulose | 0.8 |
| Silicone oil 360MED Fluid 12.500 cSt. | 2 |
| Total | 100 |
| Area Weight [g/m²] | 80 |
| Capsaicin content [μg/cm²] | 640 |

### Preparation of the Capsaicin-Containing Coating Composition

Transcutol was initially thickened with the ethyl cellulose under stirring (100-300 rpm).

A vessel was loaded with the polysiloxane mixture and the silicone oil and stirred (100-300 rpm) at least for 5 min before the ethyl cellulose/Transcutol solution was added. After further 10 min of stirring (100-300 rpm) capsaicin was added. The mixture was then stirred at approx. 250-300 rpm until a homogeneous mixture was obtained (at least 60 min).

### Coating of the Capsaicin-Containing Coating Composition

The resulting capsaicin-containing coating composition was coated on a fluoropolymer coated polyester film (Scotchpak™ 1022). The solvent was removed at room temperature for about 20-30 min.

The coating thickness was chosen such that removal of the solvent results in an area weight of the capsaicin-containing layer of about 80 g/m².

The resulting capsaicin-containing microreservoir layer was then laminated with a backing layer (polyester film, 19 μm).

### Active-Free Coating Composition

For the medical patch comprising a skin contact layer, the formulation of the active-free coating composition is summarized in Table 1.2 below. The solids %-values refer to the amounts (Amt) in % by weight.

TABLE 1.2

| Ingredient (Trade Name) | Amt [g] | Solids [%] |
|---|---|---|
| Component A of Soft Skin Adhesive Kit | 25 | 50 |
| (Liveo ™ MG 7-9900 (A)) | | |
| Component B of Soft Skin Adhesive Kit | 25 | 50 |
| (Liveo ™ MG 7-9900 (B)) | | |
| Total | 50 | 100 |

| | |
|---|---|
| Area Weight [g/m²] | 230 |

### Preparation of the Active-Free Coating Composition

Both components were weighed separately, and then component A was added to the mixing vessel followed by component B. Then the mixture was mixed at approx. 100 rpm for approx. 10 min until a homogeneous mixture of Component A and Component B was obtained.

### Coating of the Active-Free Coating Composition

Within a time frame of approx. 30 min, the resulting active-free coating composition was coated on an adhesively equipped foil. The coating temperature was set to 120° C. The resulting active-free layer was heated at this temperature for approx. 40 min.

The coating thickness was chosen such that removal of the solvents resulted in a layer thickness of the active-free (skin contact) layer of approx. 230 g/m².

The resulting active-free (skin contact) layer was laminated with a release liner (FEP, fluorinated ethylene propylene, 125 μm).

### Lamination of the Capsaicin-Containing Layer and the Active-Free (Skin Contact) Layer

The active-free (skin contact) layer was then laminated with the capsaicin-containing layer. For this purpose, the adhesively equipped foils used for the coating and drying of the layers were removed and the resulting open sides of the active-containing layer and the active-free (skin contact) layer were laminated together resulting in a capsaicin-containing self-adhesive layer structure comprising the backing layer, the capsaicin-containing layer, and the active-free (skin contact) layer, wherein the capsaicin-containing layer is attached to the backing layer, and the active-free (skin contact) layer is attached to the capsaicin-containing layer, and wherein the structure is closed by a release liner, which is attached to the active-free (skin contact) layer.

### Preparation of the Medical Patch

The individual medical patches were punched out from the capsaicin-containing self-adhesive layer structure obtained as described above. Then, the medical patches were sealed into pouches of the primary packaging material.

### Measurement of the Skin Permeation

The permeated amount of the two medical patches prepared as outlined above were determined by in vitro experiments in accordance with the OECD Guideline (adopted Apr. 13, 2004) carried out with a 10.0 ml Franz diffusion cell. Split thickness human skin from cosmetic surgeries (female abdomen, date of birth 1981) was used. A heat separated epidermis was used for all medical patches. Diecuts with an area of release of 1.171 cm² were punched from the medical patch. The capsaicin permeated amount in the receptor medium of the Franz cell (0.9% sodium chloride solution with 0.1% saline azide as antibacteriological agent) at a temperature of 32±1° C. was measured and the corresponding skin permeation rate calculated.

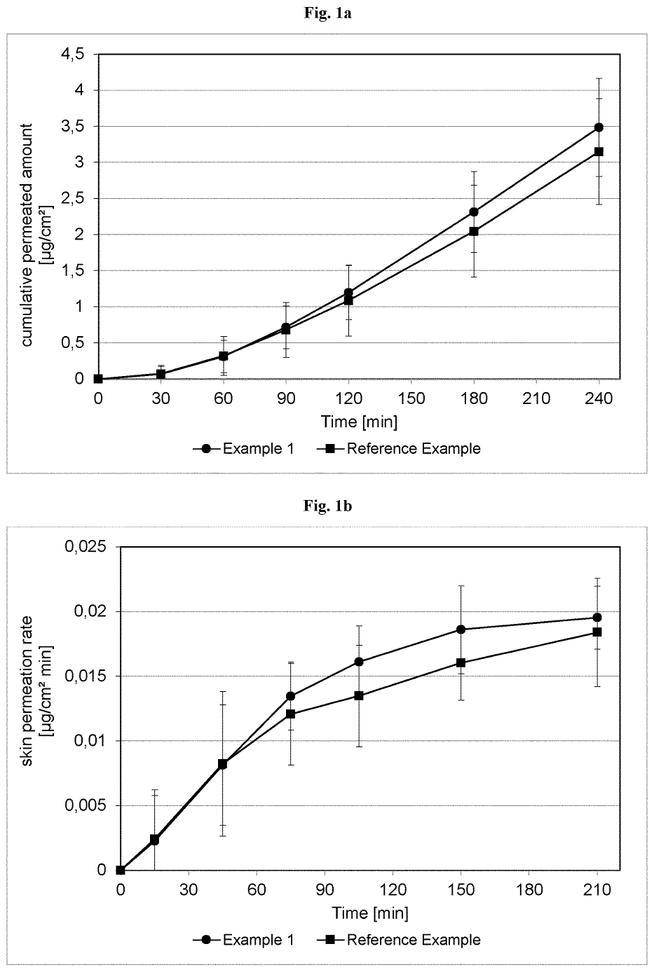

The results are shown in Table 1.3 and FIG. 1A.

TABLE 1.3
Cumulative permeated amount with SD* [μg/cm²]

| | | |
|---|---|---|
| | Patch with skin | Patch without skin |
| Elapsed | contact layer (n = 6) | contact layer (n = 6) |

| time [min] | Rate | SD | Rate | SD |
|---|---|---|---|---|
| 30 | 0.07 | 0.11 | 0.07 | 0.11 |
| 60 | 0.31 | 0.23 | 0.32 | 0.27 |
| 90 | 0.72 | 0.30 | 0.68 | 0.38 |
| 120 | 1.20 | 0.38 | 1.09 | 0.49 |
| 180 | 2.31 | 0.56 | 2.05 | 0.64 |
| 240 | 3.49 | 0.68 | 3.15 | 0.73 |

*Standard deviation was calculated based on the n-method.

The corresponding skin permeation rates (A flux rate) were calculated based on the respective permeated amount.

The results are shown in Table 1.4 and FIG. 1B.

TABLE 1.4
Skin permeation rate (Δ flux rate) with SD* [μg/cm² min]

| | | |
|---|---|---|
| | Patch with skin | Patch without skin |
| Elapsed | contact layer (n = 6) | contact layer (n = 6) |

| time [min] | Rate | SD | Rate | SD |
|---|---|---|---|---|
| 15 | 0.0023 | 0.0035 | 0.0024 | 0.0038 |
| 45 | 0.0081 | 0.0047 | 0.0082 | 0.0056 |
| 75 | 0.0135 | 0.0026 | 0.0121 | 0.0039 |
| 105 | 0.0161 | 0.0028 | 0.0135 | 0.0039 |
| 150 | 0.0186 | 0.0034 | 0.0160 | 0.0029 |
| 210 | 0.0195 | 0.0024 | 0.0184 | 0.0042 |

*Standard deviation was calculated based on the n-method.

### Exemplary Patches 2A to 2D, 3A to 3D, 4A to 4D and 5A to 5D

Based on the results obtained in Example 1 as outlined above, the concept of further patches considered to be particularly suitable, and the possible testing thereof will be described herein below.

### Active Agent-Containing Coating Composition

The formulation of possible active agent-containing coating compositions 2, 3, 4 and 5 is summarized in Table 2.1 below. The solids %-values refer to the amounts (Amt) in % by weight.

TABLE 2.1

| | active agent-containing | | | |
|---|---|---|---|---|
| | coating composition | | | |
| | 2 | 3 | 4 | 5 |
| | Solids | Solids | Solids | Solids |
| Ingredient (Trade Name) | [%] | [%] | [%] | [%] |
| Resiniferatoxin | 5-10 | 5-10 | 5-10 | 5-10 |
| Acrylic polymer (e.g. | 90-95 | — | — | — |
| acrylic adhesive in ethyl | | | | |
| acetate and n-hexane. | | | | |
| Solids content of 40.5% | | | | |
| by weight. Duro-Tak ™ | | | | |
| 87-2510) | | | | |
| Silicone-based polymer | — | 90-95 | 75-90 | — |
| (e.g. silicone adhesive | | | | |
| in n-heptane. Solids | | | | |
| content of 70% by | | | | |
| weight. Liveo ™ BIO- | | | | |
| PSA 7-4301) | | | | |
| Polyisobutylene (e.g. | — | — | — | 90-95 |
| isobutylene adhesive in | | | | |
| n-heptane, Solids | | | | |
| content of 40% by | | | | |
| weight. Oppanol ® B12/ | | | | |
| N100, 80:20) | | | | |
| Solubilizer (e.g. | — | — | 5-15 | — |
| Diethylene glycol | | | | |
| monoethyl ether. | | | | |
| Transcutol ®) | | | | |
| Total | 100 | 100 | 100 | 100 |

### Preparation and Coating of the Active Agent-Containing Coating Composition

The preparation and coating of the active agent-containing coating composition can be conducted by using methods known to the skilled person, e.g., substantially as outlined in Example 1, optionally using additional solvent during preparation of the active agent-containing coating composition, which will be evaporated when drying the coated composition.

### Active-Free Coating Composition

The formulation of the active-free coating compositions a to dis summarized in Table 2.2 below. The solids %-values refer to the amounts (Amt) in % by weight.

TABLE 2.2

| | active-free coating compositions | | | |
|---|---|---|---|---|
| | a | b | c | d |
| | Solids | Solids | Solids | Solids |
| Ingredient (Trade Name) | [%] | [%] | [%] | [%] |
| Silicone acrylic hybrid | 100 | — | — | — |
| polymer (e.g. silicone | | | | |
| acrylic hybrid pressure- | | | | |
| sensitive adhesive in n- | | | | |
| Hexane. Solids content of | | | | |
| about 50% by weight. | | | | |
| Dow-Corning ® 7-6301) | | | | |
| Silicone-based polymer | — | 100 | — | — |
| (e.g. silicone adhesive in n- | | | | |
| heptane. Solids content of | | | | |
| 70% by weight. Liveo ™ | | | | |
| BIO-PSA 7-4301) | | | | |
| Styrene-isoprene-styrene | — | — | 60 | — |
| copolymer (e.g. SIS 5229P) | | | | |
| Polyisobutylene (e.g. | — | — | — | 100 |
| isobutylene adhesive in n- | | | | |
| heptane, Solids content of | | | | |
| 40% by weight. Oppanol ® | | | | |
| B12/N100, 80:20) | | | | |
| Terpene resin | — | — | 35 | — |
| Paraffin, viscous | — | — | 5 | — |
| Total | 100 | 100 | 100 | 100 |

### Preparation and Coating of the Active-Free Coating Composition

The preparation and coating of the active agent-containing coating composition can be conducted by using methods known to the skilled person, e.g., substantially as outlined in Example 1, optionally using additional solvent during preparation of the active agent-containing coating composition, which will be evaporated when drying the coated composition.

### Lamination of the Active Agent-Containing Layer and the Active-Free (Skin Contact) Layer

The active-free (skin contact) layers can then be laminated with the active agent-containing layers obtained by using methods known to the skilled person, e.g., substantially as outlined in Example 1. The combination of formulations 2 to 5 and a to c results in patches 2a to 2d, 3a to 3d, 4a to 4d, and 5a to 5d.

### Preparation of the Medical Patch

The individual medical patches can then be punched out from the active agent-containing layer structure obtained and sealed into pouches of the primary packaging material as described above.

The invention relates in particular to the following further items:
1. A medical patch for the administration of an active agent comprising an active agent-containing layer structure, said active agent-containing layer structure comprising:
   A) a backing layer;
   B) an active agent-containing layer comprising
      (i) an active agent, and
      (ii) a polymer I;
      and
   C) a skin contact layer comprising a polymer II;
wherein the skin contact layer is an adhesive layer which is directly attached to the active agent-containing layer, and
wherein the saturation concentration of the active agent in the skin contact layer is less than 0.1% by weight.
2. Medical patch according to item 1, wherein
the saturation concentration of the active agent in the skin contact layer is less than 0.05% by weight, less than 0.02% by weight, or less than 0.01% by weight.
3. Medical patch according to item 1 or 2, wherein
the polymer II is a polymer or a mixture of polymers in which the active agent is substantially insoluble.
4. Medical patch according to any one of items 1 to 3, wherein
the polymer II is a pressure sensitive adhesive or a mixture of pressure sensitive adhesives.
5. Medical patch according to any one of items 1 to 4, wherein
the polymer II is a polymer or a mixture of polymers selected from the group consisting of silicone acrylic hybrid polymers, silicone-based polymers, silicone gel adhesives, and polymers based on natural or synthetic rubbers.
6. Medical patch according to any one of items 1 to 5, wherein
the polymer II is a polymer or a mixture of polymers selected from the group consisting of silicone-based polymers and silicone gel adhesives.
7. Medical patch according to any one of items 1 to 6, wherein
the polymer II is selected from silicone-based polymers.
8. Medical patch according to item 7, wherein
the silicone-based polymers are obtainable by polycondensation of a silanol endblocked polydimethylsiloxane with a silicate resin, preferably with a resin-to-polymer ratio of from 50:50 to 70:30 or of 55:45, 60:40 or 65:35.
9. Medical patch according to item 7 or 8, wherein
the silicone-based polymers are a mixture of pressure sensitive adhesives obtainable by polycondensation of a silanol endblocked polydimethylsiloxane with a silicate resin with a resin-to-polymer ratio of 55:45 or of 60:40.
10. Medical patch according to any one of items 7 to 9, wherein
the silicone-based polymers are a mixture of pressure sensitive adhesives with
a solution viscosity at 25° C. and about 60% solids content in heptane of 450 mPa s and/or a complex viscosity at 0.01 rad/s at 30° C. of 1×10⁸ Poise, and
a solution viscosity at 25° C. and about 60% solids content in heptane of 500 mPa s and/or a complex viscosity at 0.01 rad/s at 30° C. of 5×10⁶ Poise.
11. Medical patch according to any one of items 1 to 10, wherein
the polymer II is an amine-compatible polysiloxane.
12. Medical patch according to item 11, wherein
the amine-compatible polysiloxane is obtainable by polycondensation of a silanol endblocked polydimethylsiloxane with a silicate resin followed by at least partial trimethylsilylation of the residual silanol functionality.
13. Medical patch according to any one of items 1 to 6, wherein
the polymer II is selected from silicone gel adhesives.
14. Medical patch according to item 13, wherein
the silicone gel adhesives are obtainable by reacting a gel producing composition comprising (i) at least one alkenyl-substituted polydiorganosiloxane, (ii) at least one organosiloxane, which contains silicone-bonded hydrogen atoms, and (iii) at least one catalyst for the reaction of the SiH groups with the Si-alkenyl groups.
15. Medical patch according to item 13 or 14, wherein
the silicone gel adhesives are obtainable by reacting a gel producing composition comprising (i) a copolymer of vinylmethylsiloxane and dimethylsiloxane with (ii) methylhydrogen polysiloxane with trimethylsilyl endgroups in the presence of (iii) a platinum catalyst.
16. Medical patch according to any one of items 13 to 15, wherein
the silicone gel adhesives are silicate resin-reinforced silicone gel adhesives that contain from about 2 to about 45% by weight of at least one hydroxyl substituted silicate resin.
17. Medical patch according to any one of items 1 to 16,
with the proviso that the skin contact layer does not comprise silicone gel adhesives.
18. Medical patch according to any one of items 1 to 17, wherein
the skin contact layer comprises the polymer II in an amount of at least 95% by weight, at least 99% by weight, or in an amount of about 100% by weight, based on the total weight of the skin contact layer.
19. Medical patch according to any one of items 1 to 18, wherein
the skin contact layer comprises the active agent in an amount of less than 0.1% by weight, or less than 0.01% by weight, based on the total weight of the skin contact layer.
20. Medical patch according to any one of items 1 to 19, wherein
the active agent is an irritating active agent.
21. Medical patch according to any one of items 1 to 20, wherein
the active agent is a capsaicin analog.
22. Medical patch according to any one of items 1 to 21, wherein
the active agent is selected from the group consisting of arvanil, capsiate, civamide, dihydrocapsaicin, homocapsaicin, homodihydrocapsaicin, gingerol, nordihydrocapsaicin, olvanil, palvanil, piperine, phenylacetylrinvanil, and resiniferatoxin.
23. Medical patch according to any one of items 1 to 22, wherein
the active agent is resiniferatoxin.
24. Medical patch according to any one of items 1 to 23, wherein
with the proviso that the active agent-containing layer does not comprise capsaicin.
25. Medical patch according to any one of items 1 to 24, wherein
the active agent is present in the active agent-containing layer in an amount of at least 0.10 mg/cm², at least 0.20 mg/cm², at least 0.50 mg/cm², or at least 1.0 mg/cm².
26. Medical patch according to any one of items 1 to 25, wherein
the active agent is present in the active agent-containing layer in an amount of less than 12.0 mg/cm², less than 10.0 mg/cm², less than 6.0 mg/cm², or less than 3.0 mg/cm².
27. Medical patch according to any one of items 1 to 26, wherein
the active agent-containing layer comprises the active agent in an amount of from 0.5 to 30% by weight, from 1 to 20% by weight, from 2 to 15% by weight, or from 5 to 10% by weight, based on the total weight of the active agent-containing layer.
28. Medical patch according to any one of items 1 to 27, wherein
the active agent-containing layer comprises the polymer I in an amount of from 20 to 99% by weight, or from 60 to 90% by weight, based on the total weight of the active agent-containing layer.
29. Medical patch according to any one of items 1 to 28, wherein
the polymer I is a pressure sensitive adhesive or a mixture of pressure sensitive adhesives.
30. Medical patch according to any one of items 1 to 29, wherein
the polymer I is a polymer or a mixture of polymers selected from the group consisting of acrylic polymers, silicone acrylic hybrid polymers, silicone-based polymers, and polymers based on natural or synthetic rubbers.
31. Medical patch according to any one of items 1 to 30, wherein
the polymer I is a polymer or a mixture of polymers selected from the group consisting of silicone-based polymers and polymers based on natural or synthetic rubbers.
32. Medical patch according to any one of items 1 to 31, wherein
the polymer I is selected from silicone-based polymers.
33. Medical patch according to item 32, wherein
the silicone-based polymers are obtainable by polycondensation of a silanol endblocked polydimethylsiloxane with a silicate resin, preferably with a resin-to-polymer ratio of from 50:50 to 70:30 or of 55:45, 60:40 or 65:35.
34. Medical patch according to item 32 or 33, wherein
the silicone-based polymers are a mixture of pressure sensitive adhesives obtainable by polycondensation of a silanol endblocked polydimethylsiloxane with a silicate resin with a resin-to-polymer ratio of 55:45 or of 60:40.
35. Medical patch according to any one of items 32 to 34, wherein
the silicone-based polymers are a mixture of pressure sensitive adhesives with
a solution viscosity at 25° C. and about 60% solids content in heptane of 450 mPa s and/or a complex viscosity at 0.01 rad/s at 30° C. of 1×10⁸ Poise, and
a solution viscosity at 25° C. and about 60% solids content in heptane of 500 mPa s and/or a complex viscosity at 0.01 rad/s at 30° C. of 5×10⁶ Poise.
36. Medical patch according to any one of items 1 to 35, wherein
the polymer I is an amine-compatible polysiloxane.
37. Medical patch according to item 36, wherein
the amine-compatible polysiloxane is obtainable by polycondensation of a silanol endblocked polydimethylsiloxane with a silicate resin followed by at least partial trimethylsilylation of the residual silanol functionality.
38. Medical patch according to any one of items 1 to 37, wherein
the polymer I is selected from styrenic triblock copolymers and polyisobutylenes.
39. Medical patch according to item 38, wherein
the styrenic triblock copolymer is selected from the group consisting of styrene-ethylene-styrene (SES) block copolymers, styrene-butadiene-styrene (SBS) block copolymers, styrene-isoprene-styrene (SIS) block copolymers, styrene-ethylene/butylene-styrene (S-EB-S) block copolymers, styrene-ethylene/butylene/propylene-styrene (S-EBP-S) block copolymers, styrene-isoprene/butadiene-styrene (S—IB—S) block copolymers, and mixtures thereof.
40. Medical patch according to any one of items 1 to 39, wherein
the polymer I is a SIS block copolymer or a polyisobutylene, or a mixture thereof.
41. Medical patch according to item 40, wherein
the SIS block copolymer comprises blocks of polystyrene and of polyisoprene in a ratio of from 10:90(%) to 30:70(%).
42. Medical patch according to item 40 or 41, wherein
the SIS block copolymer consists of three blocks of polystyrene, polyisoprene and polystyrene.
43. Medical patch according to any one of items 1 to 42, wherein
the polymer I is a mixture of low-molecular weight polyisobutylene and high-molecular weight polyisobutylene.
44. Medical patch according to any one of items 1 to 43, wherein
the polymer I is different from the polymer II.
45. Medical patch according to any one of items 1 to 43, wherein
the polymer I is the same as the polymer II.
46. Medical patch according to any one of items 1 to 45, wherein
the active agent-containing layer further comprises a solubilizer.
47. Medical patch according to item 46, wherein
the solubilizer is a substance or a mixture of substances in which the active agent has a saturation concentration of at least 30% by weight.
48. Medical patch according to item 46 or 47, wherein
the solubilizer is an amphiphilic solvent.
49. Medical patch according to item 46 or 47, wherein
the solubilizer is dipropylene glycol, diethylene glycol monoethyl ether, or dimethyl isosorbide.
50. Medical patch according to any one of items 46 to 49, wherein
the active agent-containing layer comprises the solubilizer in an amount of from 1 to 40% by weight, based on the total weight of the active agent-containing layer.
51. Medical patch according to any one of items 1 to 50, wherein
wherein the active agent-containing layer and/or the skin contact layer further comprises at least one additive or excipient selected from the group consisting of crystallization inhibitors, viscosity increasing agents, fillers, substances for skincare, pH regulators, preservatives, tackifiers, softeners, stabilizers, and permeation enhancers.
52. Medical patch according to any one of items 1 to 51, wherein
the backing layer is impermeable to the active agent.
53. Medical patch according to any one of items 1 to 52, wherein
the medical patch further comprises a release liner.
54. Medical patch according to any one of items 1 to 53, wherein
the medical patch is a topical medical patch.
55. Medical patch according to any one of items 1 to 54, wherein
the medical patch is a transdermal therapeutic system.
56. Medical patch according to any one of items 1 to 55,
for use in a method of treatment, in particular for use in a method of treating pain, or for use in a method of treating neuropathic pain, or nociceptive pain, or for use in a method of treating joint pain, or cancer pain, or for use in a method of treating pain associated with an articular condition, such as arthritis.
57. Use of a medical patch according to any one of items 1 to 55,
for the manufacture of a medicament, in particular for the manufacture of a medicament for treating pain, or for the manufacture of a medicament for treating neuropathic pain, or nociceptive pain, or for the manufacture of a medicament for treating joint pain, or cancer pain, or for the manufacture of a medicament for treating pain associated with an articular condition, such as arthritis.
58. A method of treatment, in particular a method of treating pain, or a method of treating neuropathic pain, or nociceptive pain, or a method of treating joint pain, or cancer pain, or a method of treating pain associated with an articular condition, such as arthritis, including applying a medical patch according to any one of items 1 to 55 to the skin of a patient.
59. A method of manufacturing an active agent-containing layer structure of a medical patch according to any one of items 1 to 55, comprising the steps of:
   1) combining at least the components
      1. active agent, and
      2. polymer I,
      to obtain an active agent-containing coating composition;
   2.1) coating the active agent-containing coating composition on a first intermediate liner, or coating the active agent-containing coating composition on the backing layer; and
   2.2) drying the coated active agent-containing coating composition to form the active agent-containing layer.
60. The method according to item 59, further comprising the step of:
   2.3) laminating the active agent-containing layer obtained by drying the active agent-containing coating composition coated on the first intermediate liner with the backing layer.
61. Method according to item 59 or 60, further comprising the steps of:
   3.1) coating an active agent-free coating composition comprising at least the polymer II on a second intermediate liner, or
      coating an active agent-free coating composition comprising at least the polymer II on a release liner; and
   3.2) drying or curing the coated active agent-free coating composition to form the skin contact layer.
62. Method according to item 61, further comprising the step of:
   3.3) laminating the skin contact layer obtained by drying or curing the active agent-free coating composition coated on the second intermediate liner with a release liner.
63. Method according to any one of items 59 to 62, further comprising the step of:
   4.1) removing the first and second intermediate liner from the active agent-containing layer and the skin contact layer.
64. Method according to any one of items 59 to 63, further comprising the step of:
   4.2) laminating the open side of the skin contact layer onto the open side of the active agent-containing layer to obtain the active agent-containing layer structure.
65. A medical patch for the administration of an active agent comprising an active agent-containing self-adhesive layer structure, said active agent-containing self-adhesive layer structure comprising:
   A) a backing layer;
   B) an active agent-containing matrix layer comprising
      (i) an active agent in an amount of at least 0.5% by weight, based on the total weight of the active agent-containing matrix layer,
      (ii) a polymer I selected from the group consisting of silicone-based polymers and polymers based on natural or synthetic rubbers, and
      (iii) a solubilizer;
      and
   C) a skin contact layer comprising a polymer II;
wherein the skin contact layer is an adhesive layer which is directly attached to the active agent-containing layer, and
wherein the saturation concentration of the active agent in the skin contact layer is less than 0.1% by weight.

1. A medical patch for the administration of an active agent comprising an active agent-containing layer structure, said active agent-containing layer structure comprising:
A) a backing layer;
B) an active agent-containing layer comprising
(i) an active agent, and
(ii) a polymer I;
and
C) a skin contact layer comprising a polymer II;
wherein the skin contact layer is an adhesive layer which is directly attached to the active agent-containing layer, and
wherein the saturation concentration of the active agent in the skin contact layer is less than 0.1% by weight.

2. Medical patch according to claim 1, wherein
the saturation concentration of the active agent in the skin contact layer is less than 0.05% by weight, less than 0.02% by weight, or less than 0.01% by weight.

3. Medical patch according to claim 1 or 2, wherein
the polymer II is a polymer or a mixture of polymers selected from the group consisting of silicone acrylic hybrid polymers, silicone-based polymers, silicone gel adhesives, and polymers based on natural or synthetic rubbers, in particular from the group consisting of silicone-based polymers and silicone gel adhesives.

4. Medical patch according to any one of claims 1 to 3, wherein
the skin contact layer comprises the polymer II in an amount of at least 95% by weight, at least 99% by weight, or in an amount of about 100% by weight, based on the total weight of the skin contact layer.

5. Medical patch according to any one of claims 1 to 4, wherein
the skin contact layer comprises the active agent in an amount of less than 0.1% by weight, or less than 0.01% by weight, based on the total weight of the skin contact layer.

6. Medical patch according to any one of claims 1 to 5, wherein
the active agent is an irritating active agent.

7. Medical patch according to any one of claims 1 to 6, wherein
the active agent is a capsaicin analog.

8. Medical patch according to any one of claims 1 to 7, wherein
the active agent is selected from the group consisting of arvanil, capsiate, civamide, dihydrocapsaicin, homocapsaicin, homodihydrocapsaicin, gingerol, nordihydrocapsaicin, olvanil, palvanil, piperine, phenylacetylrinvanil, and resiniferatoxin, in particular the active agent is resiniferatoxin.

9. Medical patch according to any one of claims 1 to 8, wherein
the active agent is present in the active agent-containing layer in an amount of at least 0.10 mg/cm², at least 0.20 mg/cm², at least 0.50 mg/cm², or at least 1.0 mg/cm², and/or
the active agent is present in the active agent-containing layer in an amount of less than 12.0 mg/cm², less than 10.0 mg/cm², less than 6.0 mg/cm², or less than 3.0 mg/cm².

10. Medical patch according to any one of claims 1 to 9, wherein
the active agent-containing layer comprises the active agent in an amount of from 0.5 to 30% by weight, from 1 to 20% by weight, from 2 to 15% by weight, or from 5 to 10% by weight, based on the total weight of the active agent-containing layer.

11. Medical patch according to any one of claims 1 to 10, wherein
the active agent-containing layer comprises the polymer I in an amount of from 20 to 99% by weight, or from 60 to 90% by weight, based on the total weight of the active agent-containing layer.

12. Medical patch according to any one of claims 1 to 11, wherein
the polymer I is a polymer or a mixture of polymers selected from the group consisting of acrylic polymers, silicone acrylic hybrid polymers, silicone-based polymers, and polymers based on natural or synthetic rubbers, in particular from the group consisting of silicone-based polymers and polymers based on natural or synthetic rubbers.

13. Medical patch according to any one of claims 1 to 12, wherein
the active agent-containing layer further comprises a solubilizer, in particular in an amount of from 1 to 40% by weight, based on the total weight of the active agent-containing layer.

14. Medical patch according to any one of claims 1 to 13, wherein
the solubilizer is a substance or a mixture of substances in which the active agent has a saturation concentration of at least 30% by weight, and/or
the solubilizer is dipropylene glycol, diethylene glycol monoethyl ether, or dimethyl isosorbide.

15. Medical patch according to any one of claims 1 to 14, wherein
the medical patch is a topical medical patch or a transdermal therapeutic system.

16. Medical patch according to any one of claims 1 to 15,
for use in a method of treatment, in particular for use in a method of treating pain, or for use in a method of treating neuropathic pain, or nociceptive pain, or for use in a method of treating joint pain, or cancer pain, or for use in a method of treating pain associated with an articular condition, such as arthritis.

17. Use of a medical patch according to any one of claims 1 to 15,
for the manufacture of a medicament, in particular for the manufacture of a medicament for treating pain, or for the manufacture of a medicament for treating neuropathic pain, or nociceptive pain, or for the manufacture of a medicament for treating joint pain, or cancer pain, or for the manufacture of a medicament for treating pain associated with an articular condition, such as arthritis.

18. A method of treatment, in particular a method of treating pain, or a method of treating neuropathic pain, or nociceptive pain, or a method of treating joint pain, or cancer pain, or a method of treating pain associated with an articular condition, such as arthritis,
including applying a medical patch according to any one of claims 1 to 15 to the skin of a patient.

19. A method of manufacturing an active agent-containing layer structure of a medical patch according to any one of claims 1 to 15, comprising the steps of:
1) combining at least the components
1. active agent, and
2. polymer I,
to obtain an active agent-containing coating composition;
2.1) coating the active agent-containing coating composition on a first intermediate liner, or coating the active agent-containing coating composition on the backing layer;
2.2) drying the coated active agent-containing coating composition to form the active agent-containing layer; and
2.3) optionally laminating the active agent-containing layer obtained by drying the active agent-containing coating composition coated on the first intermediate liner with the backing layer.

20. Method according to claim 19, further comprising the steps of:
3.1) coating an active agent-free coating composition comprising at least the polymer II on a second intermediate liner, or
coating an active agent-free coating composition comprising at least the polymer II on a release liner;
3.2) drying or curing the coated active agent-free coating composition to form the skin contact layer; and
3.3) optionally laminating the skin contact layer obtained by drying or curing the active agent-free coating composition coated on the second intermediate liner with a release liner.

21. Method according to claim 19 or 20, further comprising the step of:
4.1) optionally removing the first and second intermediate liner from the active agent-containing layer and the skin contact layer; and
4.2) laminating the open side of the skin contact layer onto the open side of the active agent-containing layer to obtain the active agent-containing layer structure.
